SEQUENCE LISTING (2 sequences)

SEQ ID NO: 1 - protein, 165 aa, Synthetic construct [sequence omitted]

SEQ ID NO: 2 - protein, 166 aa, Synthetic construct
PCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

(19) United States
(12) Patent Application Publication
(10) Pub. No.: US 2026/0109749 A1
(43) Pub. Date: Apr. 23, 2026

(54) Mixture Of Interferon Alpha 2B With Other Modified Sequences Thereof

(71) Applicant: PharmaEssentia Corporation, Taipei (TW)

(72) Inventors: Ko-Chung Lin, Lexington, MA (US); Ming-Pin Hsu, Taipei (TW); Yu-Kuei Tsai, Taipei (TW); Yi-Te Yo, Taipei (TW); Jiing-Guang Chuang, Taipei (TW); Shin-Jye Lee, Taipei (TW)

(73) Assignee: PharmaEssentia Corporation, Taipei (TW)

(21) Appl. No.: 19/361,468

(22) Filed: Oct. 17, 2025

Related U.S. Application Data
(60) Provisional application No. 63/709,864, filed on Oct. 21, 2024.
(60) Provisional application No. 63/709,866, filed on Oct. 21, 2024.
(60) Provisional application No. 63/709,872, filed on Oct. 21, 2024.
(60) Provisional application No. 63/709,881, filed on Oct. 21, 2024.
(60) Provisional application No. 63/709,888, filed on Oct. 21, 2024.

(51) Int. Cl. C07K 14/56; A61K 38/00; C12P 21/02

(52) U.S. Cl. CPC C07K 14/56; C12P 21/02; A61K 38/00

(57) ABSTRACT

A composition and a new method of manufacturing and producing such composition including modified interferon alpha 2 b with deamidated amino acids is disclosed herein.

### CROSS-REFERENCE TO RELATED APPLICATIONS

The present application claims the benefit [under 35 U.S.C. § 119] to U.S. Provisional Patent Application Ser. Nos. 63/709,888, 63/709,864, 63/709,866, 63/709,872, and 63/709,881 that are all filed on Oct. 21, 2024, the contents of which are incorporated herein by reference in their entirety.

### SEQUENCE LISTING

This application is being filed electronically via EFS-Web and includes an electronically submitted sequence listing in .xml format in ST.26 format. The .xml file contains a sequence listing entitled “043-PCT-SEQ-Listing xml” created on Jun. 6, 2025, and having a size of 2,976 bytes and 4,096 bytes on disk. The sequence listing contained in this .xml file is part of the specification and is herein incorporated by reference in its entirety.

### TECHNICAL FIELD

The disclosure relates to the field of biomedicine, in particular to a new human interferon alpha 2b with amino acids deamidation, method of making thereof, and/or methods of detecting the use of such new method of making thereof.

### BACKGROUND

Interferon (IFN) was originally described more than 40 years ago as a substance that is produced upon stimulation of cells by viruses, and that has the ability to protect cells from infection with viruses of any kind, that is to interfere with viral infections. The antiviral activity of IFN was thus found to be non-specific, a fact that led to the idea that IFN might be used therapeutically against viral infections. The revolution that antibiotics had meant for bacterial infections, it was reasoned, might be paralleled by therapeutic use of IFN in viral infections. The human version is referred to as “hIFN”.

The optimism regarding the potential of IFN as an antiviral therapeutic agent has not been fulfilled and for various reasons it is not until during the last decade that IFN has been established as a potent antiviral agent in chronic viral infections. In parallel with being the object of antiviral research, however, IFN has also been studied with regard to its anti-tumor properties, and it is today becoming a standard treatment in certain malignant diseases.

Although IFN was initially thought to be a single entity, later research has shown that there are multiple molecular species of IFN. Thus, there are three main classes of human IFN: s called alpha, beta and gamma interferons (IFN-alpha, IFN-beta and IFN-gamma) and a minor class called omega-IFN (IFN-(2). There are 13 genes, two of which are identical, for IFN-alpha, of which there are thus 12 subtypes, but only one gene, and no subtypes for each of IFN-beta and IFNg. IFN-alpha subtypes consist of 165 or 166, IFN-beta of 165 and IFN-gamma of 142 amino acid residues. IFN-alpha and IFN-beta were formerly called type I interferons and IFN-gamma type II or immune IFN.

The reason why there are so many subtypes of IFN-alpha remain enigmatic. However, the various subtypes of IFN-alpha vary markedly regarding their biological activities. Thus, for instance, the most pronounced antiviral activity on a molar basis is found in IFN-alpha-8, and IFN-alpha-1 has certain immunological activities that are absent among other subtypes. It therefore seems plausible that the different IFN-alpha subtypes are indeed separate cytokines which share some activities, notably the antiviral capacity, but otherwise have different functional profiles.

One difference between IFN-alpha 2a and IFN-alpha 2b is the amino acid present at position 23. IFN-alpha 2a has a lysine at that position 23 while IFN-alpha 2b has arginine. For asparagine (Asn) and/or glutamine (Gln) deamidation, are known to occur in IFN. For IFN-alpha 2a, and it can drastically decrease its potency and/or anti-viral activity (up to e.g., 120 fold), even without structural change (See Bandi et al., Mol. Pharmaceutics, Publication Date (Web): 4 Sep. 2019).

Another example is in IFN-beta 1a, wherein Asn25 deamidation also caused major changes in molecular function, and was reported used as a tool for modulating therapeutic activity (see Lipari et al, Journal of Interferon & Cytokine Research, 42 (6) November 2022).

The present disclosure, in the contrary, discloses a new method of making a modified hIFN-alpha 2b having proline attached to the initial cysteine at the N-terminus. In addition, such modified hIFN-alpha 2b can further have deamidation in certain amino acid position(s), surprising without changing its potency. The disclosure further includes the use to detect whether such method of making was employed.

### SUMMARY

In an aspect, the present disclosure relates to a composition comprising deamidated Pro-hIFN A2b, Pro-hIFN A2b, and/or mixture thereof. In one embodiment the deamidated Pro-hIFN A2b comprising deamidation of one or more positions from Asn46, Asn157, Gln159, and Gln102, and any combinations thereof. In one aspect, the deamidation occur at position Asn46 and Asn157 of Pro-hIFN A2b; Asn46 and Gln159 of Pro-hIFN A2b; Asn46 and Gln102 of Pro-hIFN A2b; Asn157 and Gln159 of Pro-hIFN A2b; Asn157 and Gln102 of Pro-hIFN A2b; Gln159 and Gln102 of Pro-hIFN A2b; Asn46 Asn157 and Gln159 of Pro-hIFN A2b; or Asn46 Asn157 and Gln102 of Pro-hIFN A2b.

In another aspect, the deamidated Pro-hIFN A2b, Pro-hIFN A2b, and/or mixture thereof occurs on one or more positions from Gln at position 21, Gln at position 41, Asn at position 46, Gln at position 47, Asn at position 66, Asn at position 94, Gln at position 102, Gln at position 125, Gln at position 159, and/or Asn 157.

In another aspect, the deamidated Pro-hIFN A2b, Pro-hIFN A2b, and/or mixture thereof occurs on one or more positions from Gln at position 21, Gln at position 41, Gln at position 47, Asn at position 66, Asn at position 94, and/or Gln at position 125.

In another aspect, the deamidated Pro-hIFN A2b, Pro-hIFN A2b, and/or mixture thereof occurs at one or more positions from Gln at position 21, Gln at position 41, Asn at position 46, Gln at position 47, Asn at position 66, Asn at position 94, Gln at position 102, Gln at position 125, Gln at position 159, and/or Asn 157, each in various quantities.

In another aspect, the deamidated Pro-hIFN A2b, Pro-hIFN A2b, and/or mixture thereof occurs at one or more positions from Gln at position 21, Gln at position 41, Gln at position 47, Asn at position 66, Asn at position 94, and/or Gln at position 125, each in various quantities.

Yet in another aspect the amount of deamidated Pro-hIFN can include one or more of the following, where the percentage of deamidated species for Gln at position 21 is at least about 0.76%, the percentage of deamidated species for Gln at position 41 is at about least 0.02%, the percentage of deamidated species for Asn at position 46 is at least about 92%, the percentage of deamidated species for Gln at position 47 is at least about 0.15%, the percentage of deamidated species for Asn at position 66 is at least about 0.08%, the percentage of deamidated species for Asn at position 94 is at least about 0.11%, the percentage of deamidated species for Gln at position 102 is at least about 0.32%, the percentage of deamidated species for Gln at position 125 is at least about 0.10%, and/or the percentage of deamidated species for Asn at position 157 is at least about 2.59%, as compared to a reference standard.

In in another aspect the amount of deamidated Pro-hIFN can include one or more of the following, where the percentage of deamidated species for Gln at position 21 is at least about 0.12%, the percentage of deamidated species for Gln at position 41 is at about least 0.29%, the percentage of deamidated species for Asn at position 46 is at least about 61.64%, the percentage of deamidated species for Gln at position 47 is at least about 0.09%, the percentage of deamidated species for Asn at position 66 is at least about 0.06%, the percentage of deamidated species for Asn at position 94 is at least about 0.11%, the percentage of deamidated species for Gln at position 102 is at least about 2.37%, the percentage of deamidated species for Gln at position 125 is at least about 0.98%, and/or the percentage of deamidated species for Asn at position 157 is at least about 17.87%, as compared to a reference standard.

Yet in another aspect the amount of deamidated Pro-hIFN can include one or more of the following, where the percentage of deamidated species for Gln at position 21 is at least about 0.10%, the percentage of deamidated species for Gln at position 41 is at about least 0.20%, the percentage of deamidated species for Asn at position 46 is at least about 15.71%, the percentage of deamidated species for Gln at position 47 is at least about 0.08%, the percentage of deamidated species for Asn at position 66 is at least about 0.07%, the percentage of deamidated species for Asn at position 94 is at least about 0.14%, the percentage of deamidated species for Gln at position 102 is at least about 0.10%, the percentage of deamidated species for Gln at position 125 is at least about 0.06%, and/or the percentage of deamidated species for Asn at position 157 is at least about 0.54% %, as compared to a reference standard.

In another aspect, the percentage of deamidated species for Gln at position 21 is at least about 0.1%, the percentage of deamidated species for Asn at position 46 is at least about 92%, the percentage of deamidated species for Asn at position 157 is at least about 2.5% %, as compared to a reference standard.

Yet in another aspect, the quantification amount of the deamidated Pro-hIFN alpha 2b at Gln 21, Gln 41 and Gln 47 is about 1:2:0.8. In another aspect, the quantification amount of the deamidated Pro-hIFN alpha 2b at Gln 21, Gln 41 and Gln 47 is about 2:4.8:1.5. In another aspect, the quantification amount of the deamidated Pro-hIFN alpha at 2b Gln 21, Gln 41 and Gln 47 is about 38:1:8.

In an aspect, an analysis of the deamidated Pro-hIFN alpha 2b at Asn 66:Asn 46 and Gln 125 is about 0.8:1.4:3.8. Alternatively, or additionally, the deamidated Pro-hIFN alpha 2b at Asn 66:Asn 46 and Gln 125 is about 1:1.8:16. In an aspect, an analysis of the deamidated Pro-hIFN alpha 2b at Asn66:Asn 46 and Gln 125 is about 4:5.5:5.

Yet in another aspect, the quantification amount of the deamidated Pro-hIFN alpha 2b at Gln 21, Gln 41, Gln 47, Asn 66:Asn 46 and Gln 125 is about 1:2:0.8:0.8:1.4:3.8. In another aspect, the quantification amount of the deamidated Pro-hIFN alpha 2b at Gln 21, Gln 41, Gln 47, Asn 66:Asn 46 and Gln 125 is about 2:4.8:1.5:1.:1.8:16. In another aspect, the quantification amount of the deamidated Pro-hIFN alpha 2b at Gln 21, Gln 41, Gln 47, Asn 66:Asn 46 and Gln 125 is about 2:4.8:1.5:1.:1.8:16. In an aspect, the quantification amount of the deamidated Pro-hIFN alpha 2b at Gln 21, Gln 41, Gln 47, Asn 66:Asn 46 and Gln 125 is about 38:1:8:4:5.5:5.

In an aspect, the present disclosure relates to pharmaceutical formulation comprising a composition having deamidated Pro-hIFN A2b, Pro-hIFN A2b, and/or mixture thereof and a pharmaceutically acceptable carrier. In one embodiment the deamidated Pro-hIFN A2b comprising deamidation of one or more positions from Asn46, Asn157, Gln159, and Gln102, and/or any combinations thereof.

Yet in an aspect, the pharmaceutical formulation includes deamidated Pro-hIFN A2b, Pro-hIFN A2b, and/or mixture thereof occurs at one or more positions from Gln at position 21, Gln at position 41, Asn at position 46, Gln at position 47, Asn at position 66, Asn at position 94, Gln at position 102, Gln at position 159, Gln at position 125 and/or Asn 157.

In an aspect, the pharmaceutical formulation includes deamidated Pro-hIFN A2b, Pro-hIFN A2b, and/or mixture thereof occurs at one or more positions from Gln at position 21, Gln at position 41, Gln at position 47, Asn at position 66, Asn at position 94, and/or Gln at position 125.

In an aspect, the present disclosure relates to a method for effectively treating a subject in need who has myelofibrosis, polycythemia vera, essential thrombocythaemia, and/or chronic myeloid leukemia comprising administrating to the subject a therapeutically effective amount of the composition having deamidated Pro-hIFN A2b, Pro-hIFN A2b, and/or mixture thereof and a pharmaceutically acceptable carrier. In one embodiment the deamidated Pro-hIFN A2b comprising deamidation of one or more positions from Asn46, Asn157, Gln159, and Gln102, and any combinations thereof. Alternatively, or additionally, the composition includes deamidated Pro-hIFN A2b, Pro-hIFN A2b, and/or mixture thereof with one or more deamidated positions from Gln at position 21, Gln at position 41, Asn at position 46, Gln at position 47, Asn at position 66, Asn at position 94, Gln at position 102, Gln at position 125, Gln at position 159, and/or Asn 157.

In an aspect, the composition includes deamidated Pro-hIFN A2b, Pro-hIFN A2b, and/or mixture thereof with one or more deamidated positions from Gln at position 21, Gln at position 41, Gln at position 47, Asn at position 66, Asn at position 94, and/or Gln at position 125.

In another aspect, the present disclosure relates to a method for effectively treating a subject in need who has myelofibrosis, polycythemia vera, essential thrombocythaemia, and/or chronic myeloid leukemia comprising administrating to the subject a therapeutically effective amount of the pharmaceutical formulation comprising a composition having deamidated Pro-hIFN A2b, Pro-hIFN A2b, and/or mixture thereof and a pharmaceutically acceptable carrier. In one embodiment the deamidated Pro-hIFN A2b comprising deamidation of one or more positions from Asn46, Asn157, Gln159, and Gln102, and any combinations thereof. In one embodiment the myelofibrosis comprises idiopathic, primary, and/or early myelofibrosis. In one embodiment the polycythemia vera comprises high and/or low risk polycythemia vera. In an embodiment the pharmaceutical formulation is administered to the subject as an injection. In another embodiment the pharmaceutical formulation is administered to the subject subcutaneously. Alternatively, or additionally, the composition includes deamidated Pro-hIFN A2b, Pro-hIFN A2b, and/or mixture thereof with deamidation occurs on one or more positions from Gln at position 21, Gln at position 41, Asn at position 46, Gln at position 47, Asn at position 66, Asn at position 94, Gln at position 102, Gln at position 125 and/or Asn 157.

In an aspect, the composition includes deamidated Pro-hIFN A2b, Pro-hIFN A2b, and/or mixture thereof with one or more deamidated positions from Gln at position 21, Gln at position 41, Gln at position 47, Asn at position 66, Asn at position 94, and/or Gln at position 125.

In an aspect, the present disclosure relates to a method for producing the composition comprising deamidated Pro-hIFN A2b, Pro-hIFN A2b, and/or mixture thereof where the method comprising the steps of cloning a vector using DNA encoding the amino acid sequence of Pro-hIFN alpha 2b; transfecting said vector into a host cell; fermenting said host cell at a condition including at about 37° C. with dissolved oxygen at about 40%; optionally, the fermentation pH can be adjusted to an alkaline condition; inducing Pro-hIFN expression with IPTG at a concentration of about 0.8 mM; removing the initial methionine from said Pro-hIFN; refolding said Pro-hIFN protein; performing ultrafiltration on said Pro-hIFN protein; performing amino acetate precipitation on said Pro-hIFN protein; and purifying said Pro-hIFN protein. In one embodiment the deamidated Pro-hIFN A2b comprising deamidation of one or more positions from Asn46, Asn157, Gln159, and/or Gln102, and any combinations thereof. In an embodiment the method further comprises the step of detecting deamidated Pro-hIFN A2b at one or more positions from Asn46, Asn157, Gln159, and Gln102, and/or any combinations thereof. In an embodiment the method further comprises the step of quantifying said deamidated Pro-hIFN.

Yet in another aspect, the method according the immediate above, wherein the presence of deamidated Pro-hIFN A2b comprising deamidation of one or more positions at Gln at position 21, Gln at position 41, Asn at position 46, Gln at position 47, Asn at position 66, Asn at position 94, Gln at position 102, Gln at position 125, Gln at position 159, and/or Asn 157, and any combinations or amount thereof indicates that any of the present disclosure's method of making Pro-hIFN A2b, in part or in whole, was utilized. In an aspect, the method according the immediate above, wherein the presence of deamidated Pro-hIFN A2b comprising deamidation of one or more positions at Gln at position 21, Gln at position 41, Gln at position 47, Asn at position 66, Asn at position 94, Gln at position 125, and any combinations or amount thereof indicates that any of the present disclosure's method of making Pro-hIFN A2b, in part or in whole, was utilized.

In an aspect, Pro-hIFN A2b, and/or mixture thereof is analyzed and/or quantified in one or more samples obtained before, during, and/or after one or more steps in a manufacturing procedure. In a further aspect, the presence of one or more positions at Gln at position 21, Gln at position 41, Asn at position 46, Gln at position 47, Asn at position 66, Asn at position 94, Gln at position 102, Gln at position 159, Gln at position 125 and/or Asn 157, and/or any mixture thereof is analyzed and/or quantified in said products and/or said intermediates from one, two, three, or more individual batches at the end of a manufacturing procedure according the present disclosure. In an aspect, Pro-hIFN A2b, and/or mixture thereof is analyzed and/or quantified in one or more samples obtained before, during, and/or after one or more steps in a manufacturing procedure. In a further aspect, the presence of one or more positions at Gln at position 21, Gln at position 41, Gln at position 47, Asn at position 66, Asn at position 94, Gln at position 125, and/or any mixture thereof is analyzed and/or quantified in said products and/or said intermediates from one, two, three, or more individual batches at the end of a manufacturing procedure according the present disclosure.

In another aspect, additionally, or alternatively, some of the fermentation parameters to make Pro-hIFN alpha 2b can be adjusted to improve yield, as disclosed in United States Patent Application number US20250101484A1 (the contents of which are incorporated herein by reference in its entirety). For example, part of the fermentation can be performed including the steps of: carrying out seeding (time S₀) of a culture of prokaryote host cell engineered to inducibly express a recombinant protein and transferring the culture into one or more fermenters each containing a culture medium; measuring a dissolved oxygen (DO) level, an agitation rate, and a pH in the one or more fermenters; supplying a carbon source feeding solution to any of the one or more fermenters individually, whenever condition(s) (i) and/or (ii) are met: (i) said DO level exceeds above about 35% to 45% and said agitation rate exceeds about 300 to 1,000 rpm; (ii) said agitation rate exceeds about 400 to 700 rpm and said pH exceeds about 7.0 to 7.4; and supplying a nitrogen source to any of the one or more fermenters in which induction of expression of the recombinant protein has been initiated, individually, at about I₀ (time at initiation of induction) or I₁ or both; optionally, supplying a nitrogen source to any of the one or more fermenters, individually, at one or more time points from about S₅, S₆, S₇, S₈, S₉ and S₁₀. In an embodiment, said DO level exceeds above about 35% to 40% and said agitation rate exceeds about 300 to 700 rpm. In another embodiment, said agitation rate exceeds about 500 to 600 rpm and said pH exceeds about 7.0 to 7.4. Yet in another embodiment, the nitrogen source is supplied, individually, at about I₀ or I₁ or both, and at one or more time points from about S₅, S₆, S₇, S₈, S₉ and S₁₀. In an embodiment, a carbon source feeding solution is supplied at least once to at least one fermenter during the recombinant protein fermentation, wherein the carbon source feeding solution is a glucose feeding solution, fructose feeding solution, galactose feeding solution, pyruvate feeding solution, or any combination thereof. In another embodiment, a cumulative total of about 800 to 1500 g of glucose is supplied to at least one fermenter, per about 5 L fermenter capacity, by the end of the recombinant protein fermentation. Yet in another embodiment, the nitrogen source supplied to the one or more fermenters, individually, comprises potassium. In another embodiment, the nitrogen source supplied to the one or more fermenters, individually, comprises a yeast extract, peptone, soytone, urea, tryptone, or any combinations thereof. Yet in an embodiment, about 40 to 150 mL of a yeast extract solution is supplied the one or more fermenters each time, individually; optionally about 300 to 1500 mL of a yeast extract solution is supplied to the one or more fermenters each time, individually. In another embodiment, wherein the recombinant protein fermentation is performed with an inoculum ratio of about 6.0 to 8.5%. In an embodiment, said prokaryote host cell comprises an E. coli BL21, BLR-Codon Plus (DE3) cell. In another embedment, the fermentation process further comprises a step of collecting the culture of prokaryote host cell or a sample of the culture in the one or more fermenters after induction of expression of the recombinant protein, individually. In another embodiment, the fermentation process further comprising processing inclusion bodies in the collected culture of prokaryote cell or sample to obtain a crude protein extract. Yet in another embodiment, the culture or sample are collected after or at about 14 to 18. In an embodiment, the fermentation process the expressed recombinant protein in the crude protein extract, wherein the yield is about 5 to 10 g of the recombinant protein in a fermenter having about a 5 L capacity; optionally, wherein the yield is about 20 to 30 g of the recombinant protein in a fermenter having about a 40 L capacity. In an aspect, using this method produces the deamidated Pro-hIFN A2b, Pro-hIFN A2b, and/or mixture thereof includes deamidated amino acid at one or more positions from Gln at position 21, Gln at position 41, Asn at position 46, Gln at position 47, Asn at position 66, Asn at position 94, Gln at position 102, Gln at position 125 and/or Asn 157. In another aspect, the composition includes deamidated Pro-hIFN A2b, Pro-hIFN A2b, and/or mixture thereof with one or more deamidated positions from Gln at position 21, Gln at position 41, Gln at position 47, Asn at position 66, Asn at position 94, and/or Gln at position 125.

In an aspect, the present disclosure relates to a method of detecting whether a manufacturing procedure is used to produce Pro-hIFN alpha 2b in a product, said method comprises analyzing and/or quantifying the present of deamidated Pro-hIFN A2b in said product. In an embodiment the deamidated Pro-hIFN A2b comprises deamidation of one or more positions from Asn46, Asn157, Gln159, and Gln102, and any combinations thereof. Alternatively, or additionally, the detection of deamidated Pro-hIFN A2b, Pro-hIFN A2b, and/or mixture thereof includes deamidated amino acid at one or more positions from Gln at position 21, Gln at position 41, Gln at position 47, Asn at position 66, Asn at position 94, and/or Gln at position 125.

In another aspect, the present disclosure relates to the use of a composition comprising deamidated Pro-hIFN A2b, Pro-hIFN A2b, and/or mixture thereof for the preparation of a medicament for the treatment of one or more conditions including a subject having myelofibrosis, polycythemia vera, essential thrombocythaemia, and/or chronic myeloid leukemia. In one embodiment the deamidated Pro-hIFN A2b comprising deamidation of one or more positions from Asn46, Asn157, Gln159, and Gln102, and/or any combinations thereof. In an embodiment the myelofibrosis comprises idiopathic, primary, and/or early myelofibrosis. In an embodiment the polycythemia vera comprises high and/or low risk polycythemia vera. Alternatively, or additionally, the use includes deamidated Pro-hIFN A2b, Pro-hIFN A2b, and/or mixture thereof with deamidated amino acid at one or more positions from Gln at position 21, Gln at position 41, Asn at position 46, Gln at position 47, Asn at position 66, Asn at position 94, Gln at position 102, Gln at position 125 and/or Asn 157.

In an aspect, the present disclosure relates to a composition comprising deamidated Pro-hIFN A2b, Pro-hIFN A2b, and/or mixture thereof characterized in that it is used to treating one or more of condition including a subject having myelofibrosis, polycythemia vera, essential thrombocythaemia, and/or chronic myeloid leukemia. In one embodiment the deamidated Pro-hIFN A2b comprising deamidation of one or more positions from Asn46, Asn157, Gln159, and/or Gln102, and any combinations thereof. In an embodiment the myelofibrosis is idiopathic, primary, and/or early myelofibrosis. In an embodiment the polycythemia vera is high and/or low risk polycythemia vera. Alternatively, or additionally, the composition includes deamidated Pro-hIFN A2b, Pro-hIFN A2b, and/or mixture thereof with deamidated amino acid at one or more positions from Gln at position 21, Gln at position 41, Asn at position 46, Gln at position 47, Asn at position 66, Asn at position 94, Gln at position 102, Gln at position 125 and/or Asn 157.

In an aspect, not all of the deamidation species are present in a single run. In another aspect, no deamidation species are produced.

### BRIEF DESCRIPTION OF FIGURES

For a more complete understanding of the features and advantages of the present disclosure, reference is now made to the detailed description of the disclosure along with the accompanying figures and in which:

FIG. 1 denotes the amino acid sequence of the hIFN alpha 2b molecule, having 165 amino acids designated as SEQ ID NO:1.

FIG. 2 denotes the amino acid sequence of the modified hIFN alpha 2b molecule, namely Pro-hIFN, having 166 amino acids designated as SEQ ID NO:2.

FIG. 3(a) shows the typical mechanism of deamidation of Asn.

FIG. 3(b) shows the typical mechanism of deamidation of Gln.

FIG. 4 denotes elution profile of AIX purification performed.

FIG. 5 denotes peptide analysis of the modified hIFN alpha 2b molecule with deamidation.

FIG. 6 denotes the IEX HPLC enrichment of modified hIFN alpha 2b molecule.

FIG. 7 denotes the multi-cycle kinetic analysis design (SPR) of modified hIFN alpha 2b molecule (also known as “analyte”) to human IFNAR1-Fc/human IFNAR2-Fc.

### DETAIL DESCRIPTIONS

While the making and using of various embodiments of the present disclosure are discussed in detail below, it should be appreciated that the present disclosure provides many applicable inventive concepts that can be embodied in a wide variety of specific contexts. The specific embodiments discussed herein are merely illustrative of specific ways to make and use the disclosure and do not delimit the scope of the disclosure.

To facilitate the understanding of this disclosure, a number of terms are defined below. Terms defined herein have meanings as commonly understood by a person of ordinary skill in the areas relevant to the present disclosure. Terms such as “a”, “an” and “the” are not intended to refer to only a singular entity but include the general class of which a specific example may be used for illustration. The terminology herein is used to describe specific embodiments of the disclosure, but their usage does not delimit the disclosure, except as outlined in the claims.

The following terms, unless otherwise indicated, shall be understood to have the following meanings.

As used herein, “administering,” means oral administration, administration as a suppository, topical contact, intravenous, intraperitoneal, intramuscular, intralesional, intranasal or subcutaneous administration, or the implantation of a slow-release device e.g., a mini-osmotic pump, to the subject. Administration is by any route including parenteral, and transmucosal (e.g., oral, nasal, vaginal, rectal, or transdermal). Parenteral administration includes, e.g., intravenous, intramuscular, intra-arteriole, intradermal, subcutaneous, intraperitoneal, intraventricular, and intracranial. Moreover, where injection is to treat a tumor, e.g., induce apoptosis, administration may be directly to the tumor and/or into tissues surrounding the tumor. Other modes of delivery include, but are not limited to, the use of liposomal formulations, intravenous infusion, transdermal patches, etc.

As used herein, the term “isolated molecule” as referring to a molecule (where the molecule is, for example, a polypeptide, a polynucleotide, or an antibody) that by virtue of its origin or source of derivation (1) is not associated with naturally associated components that accompany it in its native state, (2) is substantially free of other molecules from the same source, e.g., species, cell from which it is expressed, library, etc., (3) is expressed by a cell from a different species, or (4) does not occur in nature. Thus, a molecule that is chemically synthesized, or expressed in a cellular system different from the system from which it naturally originates, will be “isolated” from its naturally associated components. A molecule also can be rendered substantially free of naturally associated components by isolation, using purification techniques well known in the art. Molecule purity or homogeneity may be assayed by a number of methods known in the art. For example, the purity of a polypeptide sample may be assayed using polyacrylamide gel electrophoresis and staining of the gel to visualize the polypeptide using techniques known in the art. For certain purposes, higher resolution may be provided by using HPLC or other means known in the art for purification.

As used herein, the terms “treating” or “treatment” or “to treat” or “alleviating” or “to alleviate” all refer to (1) therapeutic measures that cure, slow down, lessen symptoms of, and/or halt progression of a diagnosed pathologic condition or disorder and/or (2) prophylactic or preventative measures that prevent and/or slow the development of a targeted pathologic condition or disorder. Thus, those in need of treatment include those already with the disorder; those prone to have the disorder; and those in whom the disorder is to be prevented. In certain aspects, a subject is successfully “treated” according to the methods and molecules of the present disclosure if the patient shows, e.g., total, partial, or transient remission of a certain type of disorder.

As used herein, the term “substantially homogenous” means that the component can be at least about 90%, at least about 91%, at least about 92%, at least about 93%, at least about 94%, at least about 95%, at least about 96%, at least about 97%, at least about 98%, at least about 99%, and/or at least about 99.5% of the final preparation or more.

The terms “polypeptide”, “peptide” and “protein” are used interchangeably herein to refer to chains of amino acids of any length and the chain may be linear or branched. It is understood that the polypeptides can occur as single chains or associated chains.

The term “subject” refers to a living organism that can be administered by a molecule of the present disclosure and includes both humans and animals.

The term “subject in need” and “patient” are used interchangeably and refer to a living organism suffering from or prone to a condition that can be prevented or treated by administration of a molecule of the present disclosure and includes both humans and animals.

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this disclosure. One example is E. Coli.

As used herein, any concentration range, percentage range, ratio range or integer range is to be understood to include the value of any integer as indicated per se, as well as within the recited range and, when appropriate, fractions thereof (such as one tenth and one hundredth of an integer), unless otherwise indicated.

As used herein, an “effective dosage”, “effective therapeutic amount”, or “effective amount” of drug, compound, or composition is an amount sufficient to affect any one or more beneficial or desired results. In more specific aspects, an effective amount prevents, alleviates, ameliorates symptoms of disease, and/or prolongs the survival of the subject being treated. For prophylactic use, beneficial or desired results include, but not limited to eliminating or reducing the risk, lessening the severity, or delaying the outset of the disease, including biochemical, histological and/or behavioral symptoms of the disease, its complications and intermediate pathological phenotypes presenting during development of the disease. For therapeutic use, beneficial or desired results include, but not limited to clinical results such as reducing one or more symptoms of a disease such as cancer. An effective dosage can be administered in one or more administrations. For purposes of this disclosure, an effective dosage of drug, compound, or composition is an amount sufficient to accomplish prophylactic or therapeutic treatment either directly or indirectly. As is understood in the clinical context, an effective dosage of a drug, compound, or composition may or may not be achieved in conjunction with another drug, compound, or pharmaceutical composition. Thus, an “effective dosage” or “effective amount” may be considered in the context of administering one or more therapeutic agents, and a single agent may be considered to be given in an effective amount if, in conjunction with one or more other agents, a desirable result may be or is achieved.

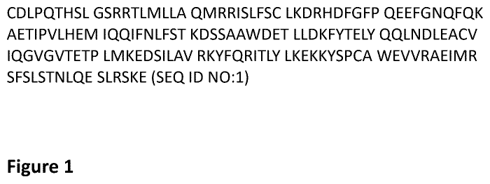

As used herein, the term “hIFN alpha 2b” or “hIFN-A2b” refer to the human version of interferon having category alpha in class 2b, wherein the amino acid sequence is in FIG. 1 (SEQ ID NO: 1).

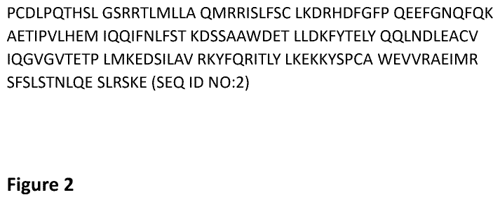

As used herein, the term “Pro-hIFN-A2b” refer to modified hIFN alpha 2b having the amino acid sequence denoted in FIG. 2 (SEQ ID NO:2).

As used herein, the term “per dose”, “dosage”, or “dose” means administering a given numeric amount of drug to a subject. Per dose can be administered in separate injections at about the same time and/or different time, so long as a subject receives the drug amount.

As used herein, the term “per injection” means administering the entire numeric amount of drug to a subject for a given dose in a single injection.

As used herein, “substantially” or “substantially all” means more than about 50%, 60%, 70%, 80%, 90%, 92.5%, 95%, 97.5%, 98%, 99%, 99.5%, 99.95% or 100% of a molecule.

As used herein, “vector” or “expression vector” means a construct, which is capable of delivering, and expressing one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

As used herein, “pharmaceutically acceptable carrier” or “pharmaceutical acceptable excipient” includes any material which, when combined with an active ingredient, allows the ingredient to retain biological activity or stability and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline (PBS) or normal (0.9%) saline. Compositions comprising such carriers are formulated by known methods.

The term “intradermal administration”, “i.d.”, or “administered intradermally,” in the context of administering a substance to a mammal including a human, refers to the delivery of the substance into the dermis layer of the skin of the mammal. The skin of a mammal is composed of an epidermis layer, a dermis layer, and a subcutaneous layer. The epidermis is the outer layer of the skin. The dermis, which is the middle layer of the skin, contains nerve endings, sweat glands and oil (sebaceous) glands, hair follicles, and blood vessels. The subcutaneous layer is made up of fat and connective tissue that houses larger blood vessels and nerves. In contrast in intradermal administration, “s.c.”, or “subcutaneous administration”, refers to the administration of a substance into the subcutaneous layer and “topical administration” refers to the administration of a substance onto the surface of the skin.

As used herein, “about” mean within an acceptable error range for the particular value as determined by one of ordinary skill in the art, which will depend in part on how the value is measured or determined, i.e., the limitations of the measurement system. For example, “about” can mean within 1 or more than 1 standard deviation per the practice in the art. Alternatively, “about” can mean a range of up to 5%, 7.5%, 10%, 12.5%, 15%, 17.55, 20%, 22.5% 25%, 27.5%, 30%, 32.5%, 35%, 37.5%, or 40% of difference in either direction (positive or negative) compared to a reference value. Furthermore, particularly with respect to biological systems or processes, the terms can mean up to an order of magnitude or up to 1, 2, 3, 4, or 5-folds of a value. When particular values are provided in the application and claims, unless otherwise stated, the meaning of “about” should be assumed to be within an acceptable error range for that particular value. Reference to “about” a value or parameter herein includes (and describes) embodiments that are directed to that value or parameter per se. For example, description referring to “about X” includes description of “X”. Numeric ranges are inclusive of the numbers defining the range.

In an embodiment, the present disclosure includes a biologics molecule having 165 amino acids, and one particular example is denoted in FIG. 1 as SEQ ID No.: 1. The biologics molecule is known as IFN alfa-2b.

In another embodiment, the present disclosure includes a biologics molecule having 166 amino acids, named Pro-IFN alfa-2b as shown in FIG. 2. In particular, it is bacterially produced with extra N-terminal Methionine (Met) with Proline (Pro) added, and the Met is removed via method known in the art.

Yet in an embodiment, one or more amino acids of Pro-IFN alfa-2b can undergo deamidation. Yet in another embodiment, the present composition can contain deamidated Pro-IFN alfa-2b, Pro-IFN alfa-2b, and/or mixture thereof.

Deamidation of asparagine and/or glutamine alters protein structures and affects the chemical and biological properties of proteins. Deamidation is a non-enzymatic reaction during which the covalent amide functional group is converted to a carboxylic acid. In proteins and peptides, deamidation can happen on asparagine (Asn) and/or glutamine (Gln) residues as a post-translational modification (PTM) to produce aspartic acid (Asp) and/or glutamic acid (Glu).

In one embodiment the present disclosure relates to deamidated Pro-hIFN A2b comprising deamidation of one or more positions from Asn46, Asn157, Gln159, and Gln102, and/or any combinations thereof. In an embodiment, the mere existence of one or more of the deamidated species indicated in the present disclosure, shows that the present disclosure's method, in part or in whole was utilized.

In an embodiment the present disclosure relates to deamidated Pro-hIFN A2b comprising deamidation of one or more positions from Gln21, Gln41, Gln47, Asn66, Asn94, Gln125, and/or any combinations thereof. In an embodiment, the mere existence of one or more of the deamidated species indicated in the present disclosure, shows that the present disclosure's method, in part or in whole was utilized.

In another embodiment, the deamidation can occur at position Asn46 and Asn157 of Pro-hIFN A2b; Asn46 and Gln159 of Pro-hIFN A2b; Asn46 and Gln 102 of Pro-hIFN A2b; Asn157 and Gln159 of Pro-hIFN A2b; Asn157 and Gln102 of Pro-hIFN A2b; Gln159 and Gln102 of Pro-hIFN A2b; Asn46 Asn157 and Gln159 of Pro-hIFN A2b; and/or Asn46 Asn157 and Gln102 of Pro-hIFN A2b. In an embodiment, the mere existence of one or more of the deamidated species indicated in the present disclosure, shows that the present disclosure's method, in part or in whole was utilized.

Yet in another embodiment, the deamidated Pro-hIFN A2b comprising deamidation on one or more positions from Gln at position 21, Gln at position 41, Gln at position 47, Asn at position 66, Asn at position 94, and/or Gln at position 125. In an embodiment, the mere existence of one or more of the deamidated species indicated in the present disclosure, shows that the present disclosure's method, in part or in whole was utilized.

In one embodiment the present disclosure relates to deamidated Pro-hIFN A2b, Pro-hIFN A2b, and/or mixture thereof comprising deamidation of one or more positions from Asn46, Asn157, Gln159, and/or Gln102, and any combinations thereof. In an embodiment, the mere existence of one or more of the deamidated species indicated in the present disclosure, shows that the present disclosure's method, in part or in whole was utilized.

In another embodiment, the deamidated Pro-hIFN A2b, Pro-hIFN A2b, and/or mixture thereof can have deamidation occur at position Asn46 and Asn157 of Pro-hIFN A2b; Asn46 and Gln159 of Pro-hIFN A2b; Asn46 and Gln102 of Pro-hIFN A2b; Asn157 and Gln159 of Pro-hIFN A2b; Asn157 and Gln102 of Pro-hIFN A2b; Gln159 and Gln102 of Pro-hIFN A2b; Asn46 Asn157 and Gln159 of Pro-hIFN A2b; and/or Asn46 Asn157 and Gln102 of Pro-hIFN A2b. In an embodiment, the mere existence of one or more of the deamidated species indicated in the present disclosure, shows that the present disclosure's method, in part or in whole was utilized.

Yet in another embodiment, the deamidated Pro-hIFN A2b, Pro-hIFN A2b, and/or mixture thereof comprising deamidation on one or more positions from Gln at position 21, Gln at position 41, Asn at position 46, Gln at position 47, Asn at position 66, Asn at position 94, Gln at position 102 Gln at position 125 and/or Asn 157. In an embodiment, the mere existence of one or more of the deamidated species indicated in the present disclosure, shows that the present disclosure's method, in part or in whole was utilized.

Yet in another embodiment, the quantification amount of the deamidated Pro-hIFN alpha 2b at Gln 21, Gln 41 and Gln 47 ratio is about 1:2:0.8. In another embodiment, the quantification amount of the deamidated Pro-hIFN alpha 2b at Gln 21, Gln 41 and Gln 47 ratio is about 2:4.8:1.5. In another embodiment, the quantification amount of the deamidated Pro-hIFN alpha 2b at Gln 21, Gln 41 and Gln 47 ratio is about 38:1:8. In an embodiment, the mere existence of one or more of the deamidated species indicated in the present disclosure, or in any of the quantity/ratio stated in the present disclosure, shows that the present disclosure's method, in part or in whole was utilized.

In an embodiment, an analysis of the deamidated Pro-hIFN alpha 2b at Asn 66, Asn 46 and Gln 125 ratio is about 0.8:1.4:3.8. Alternatively, or additionally, the deamidated Pro-hIFN alpha 2b at Asn 66, Asn 46 and Gln 125 ratio is about 1:1.8:16. In an embodiment, an analysis of the deamidated Pro-hIFN alpha 2b at Asn66, Asn96 and Gln 125 ratio is about 4:5.5:5. In an embodiment, the mere existence of one or more of the deamidated species indicated in the present disclosure, or in any of the quantity/ratio stated in the present disclosure, shows that the present disclosure's method, in part or in whole was utilized.

Yet in another embodiment, the quantification amount of the deamidated Pro-hIFN alpha 2b at Gln 21, Gln 41, Gln 47, Asn 66, Asn 46 and Gln 125 ratio is about 1:2:0.8:0.8:1.4:3.8. In another embodiment, the quantification amount of the deamidated Pro-hIFN alpha 2b at Gln 21, Gln 41, Gln 47, Asn 66, Asn 46 and Gln 125 ratio is about 2:4.8:1.5:1.:1.8:16. In another embodiment, the quantification amount of the deamidated Pro-hIFN alpha 2b at Gln 21, Gln 41, Gln 47, Asn 66, Asn 46 and Gln 125 ratio is about 2:4.8:1.5:1.:1.8:16. In an embodiment, the quantification amount of the deamidated Pro-hIFN alpha 2b at Gln 21, Gln 41, Gln 47, Asn 66, Asn 46 and Gln 125 ratio is about 38:1:8:4:5.5:5. In an embodiment, the mere existence of one or more of the deamidated species indicated in the present disclosure, or in any of the quantity/ratio stated in the present disclosure, shows that the present disclosure's method, in part or in whole was utilized.

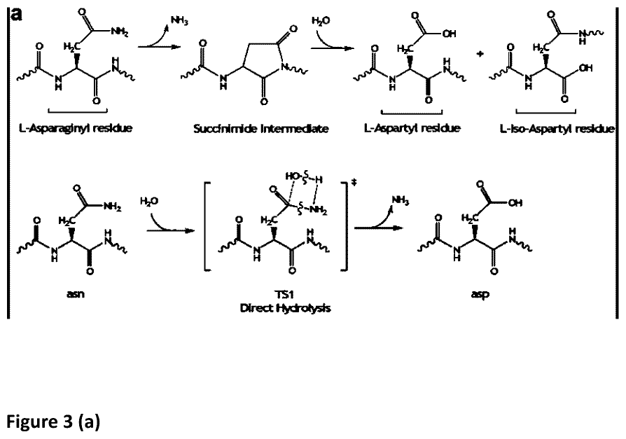

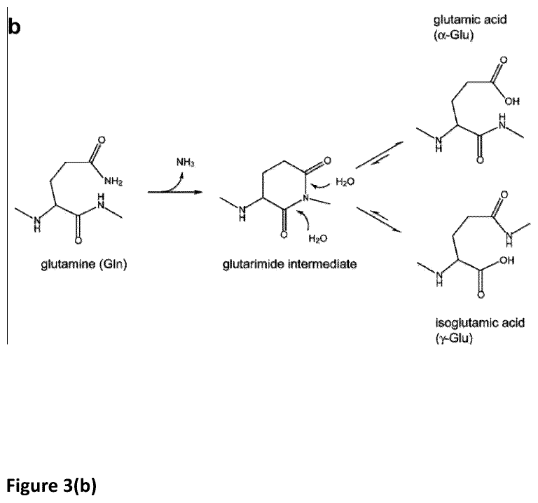

Under alkaline conditions, deamidation of Asn proceeds via a succinimide intermediate formed by the nucleophilic attack of the side chain carbonyl carbon by the backbone amine (see FIG. 3(a)). The succinimide ring is then hydrolyzed at either the α- or β-carbonyl groups to produce isoAsp and Asp products. Under acidic conditions, Asn usually deamidates by direct hydrolysis via acid catalysis.

Deamidation of Gln forms by direct hydrolysis at acidic pH and occurs via a glutarimide intermediate at alkaline pH (see FIG. 3(b)).

The nucleotide sequences encoding such amino acid can vary such as using codon optimization, if needed, and depending on which host cell is used. Such method is known in the art. The nucleotide sequence can be cloned into a vector and transfect into a bacterial host cell for protein expression by methods known in the art.

Previously, a method to generate Pro-IFN A2b is known. See U.S. Pat. No. 8,106,160, the content of which are incorporated herein by reference in their entirety. In U.S. Pat. No. 8,106,160, no deamidated amino acid of Pro-IFN A2b was reported. Here, in an embodiment, the method of making is not identical and deamidated amino acid of Pro-hIFN alpha 2b can be produced, but not necessarily all the time. In an embodiment, the presence of deamidated amino acid of Pro-hIFN alpha 2b species can be detected 90%, 91%, 92%, 93%, 94%, 95%, 96%, 97%, 98%, 99%, 99.5%, 99.9% and/or 99.99% over time.

In an embodiment, the present disclosure provides new methods of generating and making deamidated Pro-hIFN A2b, Pro-hIFN A2b, and/or mixture thereof.

Briefly, and in general, DNA encoding amino acid of SEQ ID No:2 with is constructed in a vector, and such vectors encoding Pro-hIFN are transfected to host cells such as E. Coli. Then, the host cells are first inoculated, and cultured in a fermenter. Optionally, the fermentation pH can be adjusted to an alkaline condition. In an embodiment, the alkaline condition can be about pH 6.0, 6.5, 7.0, 7.1, 7.2, 7.3, 7.4, 7.5, 7.6, 7.7, 7.8, 7.9, 8.0, 8.1, 8.2, 8.3, 8.4, or 8.5. Induction is conducted with IPTG (Isopropyl β-D-1-thiogalactopyranoside) to induce protein product in the host cell, and then the cells are harvested. The cell pellet is then stored. Then, the cell pellet is homogenized to obtain a crude lysate which contains the protein Pro-hIFN. The initial methionine, which is added on by E. Coli, is then removed, and crude lysate protein is then refolded by method known in the art, and clarified to obtain crude refolded protein solution. The crude solution is then concentrated and subjected to filtration, chromatography, and/or hydrophobic interaction chromatography to obtain deamidated Pro-hIFN A2b, Pro-hIFN A2b, and/or mixture thereof. In an embodiment, the mere existence of one or more of the deamidated species indicated in the present disclosure (e.g., Asn45, Asn157, Gln102, and/or Gln159), shows that the present disclosure's method, in part or in whole was utilized. In an embodiment, the mere existence of one or more of the deamidated species indicated in the present disclosure (e.g., Asn46, Asn157, Gln102, Gln159, Gln21, Gln41, Gln47, Asn66, Asn94 and/or Gln125), shows that the present disclosure's method, in part or in whole was utilized. In another embodiment, the mere existence of one or more of the deamidated species indicated in the present disclosure (e.g., Gln21, Gln41, Gln47, Asn66, Asn94 and/or Gln125), shows that the present disclosure's method, in part or in whole was utilized.

In another embodiment, additionally, or alternatively, some of the fermentation parameters can be adjusted to improve yield, as disclosed in United States Patent Application number US20250101484A1. The contents of which are incorporated herein by reference in its entirety. For example, part of the fermentation can be performed including the steps of: carrying out seeding (time S₀) of a culture of prokaryote host cell engineered to inducibly express a recombinant protein and transferring the culture into one or more fermenters each containing a culture medium; measuring a dissolved oxygen (DO) level, an agitation rate, and a pH in the one or more fermenters; supplying a carbon source feeding solution to any of the one or more fermenters individually, whenever condition(s) (i) and/or (ii) are met: (i) said DO level exceeds above about 35% to 45% and said agitation rate exceeds about 300 to 1,000 rpm; (ii) said agitation rate exceeds about 400 to 700 rpm and said pH exceeds about 7.0 to 7.4; and supplying a nitrogen source to any of the one or more fermenters in which induction of expression of the recombinant protein has been initiated, individually, at about I₀ (time at initiation of induction) or I₁ or both; optionally, supplying a nitrogen source to any of the one or more fermenters, individually, at one or more time points from about S₅, S₆, S₇, S₈, S₉ and S₁₀. In an embodiment, said DO level exceeds above about 35% to 40% and said agitation rate exceeds about 300 to 700 rpm. In another embodiment, said agitation rate exceeds about 500 to 600 rpm and said pH exceeds between about 7.0 to 7.4. Yet in another embodiment, the nitrogen source is supplied, individually, at about I₀ or I₁ or both, and at one or more time points from about S₅, S₆, S₇, S₈, S₉ or S₁₀. In an embodiment, a carbon source feeding solution is supplied at least once to at least one fermenter during the recombinant protein fermentation, wherein the carbon source feeding solution is a glucose feeding solution, fructose feeding solution, galactose feeding solution, pyruvate feeding solution, or any combination thereof. In another embodiment, a cumulative total of about 800 to 1500 g of glucose is supplied to at least one fermenter, per about 5 L fermenter capacity, by the end of the recombinant protein fermentation. Yet in another embodiment, the nitrogen source supplied to the one or more fermenters, individually, comprises potassium. In another embodiment, the nitrogen source supplied to the one or more fermenters, individually, comprises a yeast extract, peptone, soytone, urea, tryptone, or any combinations thereof. Yet in an embodiment, about 40 to 150 mL of a yeast extract solution is supplied the one or more fermenters each time, individually; optionally about 300 to 1500 mL of a yeast extract solution is supplied to the one or more fermenters each time, individually. In another embodiment, wherein the recombinant protein fermentation is performed with an inoculum ratio of about 6.0 to 8.5%. In an embodiment, said prokaryote host cell comprises an E. coli BL21, BLR-Codon Plus (DE3) cell. In another embedment, the fermentation process further comprises a step of collecting the culture of prokaryote host cell or a sample of the culture in the one or more fermenters after induction of expression of the recombinant protein, individually. In another embodiment, the fermentation process further comprising processing inclusion bodies in the collected culture of prokaryote cell or sample to obtain a crude protein extract. Yet in another embodiment, the culture or sample are collected after or at about 14 to 18. In an embodiment, the fermentation process for the expressed recombinant protein in the crude protein extract, wherein the yield is about 5 to 10 g of the recombinant protein in a fermenter can be about a 5 L capacity; optionally, wherein the yield is about 20 to 30 g of the recombinant protein in a fermenter having about a 40 L capacity. In an embodiment, the detection of the existence of any amount of the deamidated species indicated in the present disclosure, shows that the any of the present disclosure's method, or present paragraph's method, was utilized in part or in whole. In an embodiment, the mere existence of one or more of the deamidated species indicated in the present disclosure (e.g., Asn45, Asn157, Gln102, and/or Gln159), shows that the present paragraph's method, in part or in whole was utilized. In an embodiment, the mere existence of one or more of the deamidated species indicated in the present disclosure (e.g., Asn46, Asn157, Gln102, Gln159, Gln21, Gln41, Gln47, Asn66, Asn94 and/or Gln125), shows that the present paragraph's method, in part or in whole was utilized. In another embodiment, the mere existence of one or more of the deamidated species indicated in the present disclosure (e.g., Gln21, Gln41, Gln47, Asn66, Asn94 and/or Gln125), shows that the present paragraph's method, in part or in whole was utilized.

In an embodiment, the amount of deamidated Pro-hIFN can include one or more of the following, where the percentage of deamidated species for Gln at position 21 is at least about 0.76%, the percentage of deamidated species for Gln at position 41 is at about least 0.02%, the percentage of deamidated species for Asn at position 46 is at least about 92%, the percentage of deamidated species for Gln at position 47 is at least about 0.15%, the percentage of deamidated species for Asn at position 66 is at least about 0.08%, the percentage of deamidated species for Asn at position 94 is at least about 0.11%, the percentage of deamidated species for Gln at position 102 is at least about 0.32%, the percentage of deamidated species for Gln at position 125 is at least about 0.10%, and/or the percentage of deamidated species for Asn at position 157 is at least about 2.59%. In an embodiment, the mere existence of one or more of the deamidated species indicated in the present disclosure, shows that any of the present disclosure's methods, in part or in whole, was utilized.

In in another embodiment, the amount of deamidated Pro-hIFN can include one or more of the following, where the percentage of deamidated species for Gln at position 21 is at least about 0.12%, the percentage of deamidated species for Gln at position 41 is at about least 0.29%, the percentage of deamidated species for Asn at position 46 is at least about 61.64%, the percentage of deamidated species for Gln at position 47 is at least about 0.09%, the percentage of deamidated species for Asn at position 66 is at least about 0.06%, the percentage of deamidated species for Asn at position 94 is at least about 0.11%, the percentage of deamidated species for Gln at position 102 is at least about 2.37%, the percentage of deamidated species for Gln at position 125 is at least about 0.98%, and/or the percentage of deamidated species for Asn at position 157 is at least about 17.87%. In an embodiment, the mere existence of one or more of the deamidated species indicated in the present disclosure, shows that any the present disclosure's method, in part or in whole was utilized.

Yet in another embodiment, the amount of deamidated Pro-hIFN can include one or more of the following, where the percentage of deamidated species for Gln at position 21 is at least about 0.10%, the percentage of deamidated species for Gln at position 41 is at about least 0.20%, the percentage of deamidated species for Asn at position 46 is at least about 15.71%, the percentage of deamidated species for Gln at position 47 is at least about 0.08%, the percentage of deamidated species for Asn at position 66 is at least about 0.07%, the percentage of deamidated species for Asn at position 94 is at least about 0.14%, the percentage of deamidated species for Gln at position 102 is at least about 0.10%, the percentage of deamidated species for Gln at position 125 is at least about 0.06%, and/or the percentage of deamidated species for Asn at position 157 is at least about 0.54%. In an embodiment, the mere existence of one or more of the deamidated species indicated in the present disclosure, shows that any of the present disclosure's methods, in part or in whole, was utilized.

In another embodiment, in another aspect the percentage of deamidated species for Gln at position 21 is at least about 0.1%, the percentage of deamidated species for Asn at position 46 is at least about 92%, the percentage of deamidated species for Asn at position 157 is at least about 2.5%. In an embodiment, the mere existence of one or more of the deamidated species indicated in the present disclosure, shows that any of the present disclosure's method, in part or in whole was utilized.

The composition used in the present disclosure, whether deamidated Pro-hIFN A2b, Pro-hIFN A2b, and/or mixture thereof alone, or with any combinations of other active ingredients thereof, can further comprise pharmaceutically acceptable carriers, excipients, or stabilizers, in the form of lyophilized formulations or aqueous solutions. Carriers, excipients, or stabilizers can be nontoxic to recipients at the dosages and concentrations, and can comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN (trademarked), PLURONICS (trademarked).

In an embodiment, a subject can be treated with deamidated Pro-hIFN A2b, Pro-hIFN A2b, and/or mixture thereof for a disease. In another embodiment, a disease or indication can be myelofibrosis of any subtype (e.g., idiopathic, primary, and/or early), polycythemia vera (high or low risk), essential thrombocythaemia, and/or chronic myeloid leukemia.

Yet in an embodiment, a treatment period using deamidated Pro-hIFN A2b, Pro-hIFN A2b, and/or mixture thereof can be at least about 1, 2, 3, 4, 5, 6, 7, 8, 9, 10, 11, 12, 24, 36, 42, 48, 54, 60, 66, 72, 78, 84 or more than 84 months. In some embodiments, the treatment period is about 0.1, 0.25, 0.5, 0.75, 1, 2, 3, 4, 4.5, 5, 5.5, 6, 6.5, 7, 7.5, 8, 8.5, 9, 9.5, 10 or more than 10 years. In some embodiments, the treatment period is at least about 0.1, 0.25, 0.5, 0.75, 1, 2, 3, 4, 5, 6, 7, 8, 12, 14, 15, 16, 17, 18, 19, 20, 24, 28, 32, 36, 40, 44, 48, 52, 56, 60 weeks or longer than 60 weeks.

In another embodiment, deamidated Pro-hIFN A2b, Pro-hIFN A2b, and/or mixture thereof can be administered for treating myelofibrosis of any subtype (e.g., idiopathic, primary, and/or early), polycythemia vera (high or low risk), essential thrombocythaemia, and/or chronic myeloid leukemia using between about 50 to 540 μg per dose or per injection subcutaneously, via i.d., or other routes. Alternatively, or additionally, a dose or injection of deamidated Pro-hIFN A2b, Pro-hIFN A2b, and/or mixture thereof can be administered during the treatment period ranges from about 250 to about 650 μg. The dose/injection can also be up to about 250 μg, up to about 255 μg, up to about 260 μg, up to about 265 μg, up to about 270 μg, up to about 275 μg, up to about 280 μg, up to about 285 μg, up to about 290 μg, up to about 295 μg, up to about 300 μg, up to about 305 μg, up to about 310 μg, up to about 315 μg, up to about 320 μg, up to about 325 μg, up to about 330 μg, up to about 335 μg, up to about 340 μg, up to about 345 μg, up to about 350 μg, up to about 400 μg, up to about 450 μg, up to about 500 μg, up to about 540 μg, or up to about 650 μg. Alternatively, or additionally, a dose or injection of 135 μg or 180 μg of deamidated Pro-hIFN A2b, Pro-hIFN A2b, and/or mixture thereof can be administered.

In another embodiment, deamidated Pro-hIFN A2b, Pro-hIFN A2b, and/or mixture thereof can be administered for treating myelofibrosis of any subtype (e.g., idiopathic, primary, and/or early), polycythemia vera (high or low risk), essential thrombocythaemia, and/or chronic myeloid leukemia using between about 50 to 540 μg per dose or per injection subcutaneously, via i.d., or other routes every week. Alternatively, or additionally, a dose or injection of deamidated Pro-hIFN A2b, Pro-hIFN A2b, and/or mixture thereof can be administered during the treatment period ranges from about 250 to about 650 μg. The dose/injection can also be up to about 250 μg, up to about 255 μg, up to about 260 μg, up to about 265 μg, up to about 270 μg, up to about 275 μg, up to about 280 μg, up to about 285 μg, up to about 290 μg, up to about 295 μg, up to about 300 μg, up to about 305 μg, up to about 310 μg, up to about 315 μg, up to about 320 μg, up to about 325 μg, up to about 330 μg, up to about 335 μg, up to about 340 μg, up to about 345 μg, up to about 350 μg, up to about 400 μg, up to about 450 μg, up to about 500 μg, up to about 540 μg, or up to about 650 μg every week. Alternatively, or additionally, a dose or injection of 135 μg or 180 μg of deamidated Pro-hIFN A2b, Pro-hIFN A2b, and/or mixture thereof can be administered every week.

In another embodiment, deamidated Pro-hIFN A2b, Pro-hIFN A2b, and/or mixture thereof can be administered for treating myelofibrosis of any subtype (e.g., idiopathic, primary, and/or early), polycythemia vera (high or low risk), essential thrombocythaemia, and/or chronic myeloid leukemia using between about 50 to 540 μg per dose or per injection subcutaneously, via i.d., or other routes every 2 weeks. Alternatively, or additionally, a dose or injection of deamidated Pro-hIFN A2b, Pro-hIFN A2b, and/or mixture thereof can be administered during the treatment period ranges from about 250 to about 650 μg. The dose/injection can also be up to about 250 μg, up to about 255 μg, up to about 260 μg, up to about 265 μg, up to about 270 μg, up to about 275 μg, up to about 280 μg, up to about 285 μg, up to about 290 μg, up to about 295 μg, up to about 300 μg, up to about 305 μg, up to about 310 μg, up to about 315 μg, up to about 320 μg, up to about 325 μg, up to about 330 μg, up to about 335 μg, up to about 340 μg, up to about 345 μg, up to about 350 μg, up to about 400 μg, up to about 450 μg, up to about 500 μg, up to about 540 μg, or up to about 650 μg every 2 weeks. Alternatively, or additionally, a dose or injection of 135 μg or 180 μg of deamidated Pro-hIFN A2b, Pro-hIFN A2b, and/or mixture thereof can be administered every 2 weeks.

Yet in another embodiment, deamidated Pro-hIFN A2b, Pro-hIFN A2b, and/or mixture thereof can be administered for treating myelofibrosis of any subtype (e.g., idiopathic, primary, and/or early), polycythemia vera (high or low risk), essential thrombocythaemia, and/or chronic myeloid leukemia using between about 50 to 540 μg per dose or per injection subcutaneously, via i.d., or other routes every 3 weeks. Alternatively, or additionally, a dose or injection of deamidated Pro-hIFN A2b, Pro-hIFN A2b, and/or mixture thereof can be administered during the treatment period ranges from about 250 to about 650 μg. The dose/injection can also be up to about 250 μg, up to about 255 μg, up to about 260 μg, up to about 265 μg, up to about 270 μg, up to about 275 μg, up to about 280 μg, up to about 285 μg, up to about 290 μg, up to about 295 μg, up to about 300 μg, up to about 305 μg, up to about 310 μg, up to about 315 μg, up to about 320 μg, up to about 325 μg, up to about 330 μg, up to about 335 μg, up to about 340 μg, up to about 345 μg, up to about 350 μg, up to about 400 μg, up to about 450 μg, up to about 500 μg, up to about 540 μg, or up to about 650 μg every 3 weeks. Alternatively, or additionally, a dose or injection of 135 μg or 180 μg of deamidated Pro-hIFN A2b, Pro-hIFN A2b, and/or mixture thereof can be administered every 3 weeks.

In another embodiment, deamidated Pro-hIFN A2b, Pro-hIFN A2b, and/or mixture thereof can be administered for treating myelofibrosis of any subtype (e.g., idiopathic, primary, and/or early), polycythemia vera (high or low risk), essential thrombocythaemia, and/or chronic myeloid leukemia using between about 50 to 540 μg per dose or per injection subcutaneously, via i.d., or other routes every 4 weeks. Alternatively, or additionally, a dose or injection of deamidated Pro-hIFN A2b, Pro-hIFN A2b, and/or mixture thereof can be administered during the treatment period ranges from about 250 to about 650 μg. The dose/injection can also be up to about 250 μg, up to about 255 μg, up to about 260 μg, up to about 265 μg, up to about 270 μg, up to about 275 μg, up to about 280 μg, up to about 285 μg, up to about 290 μg, up to about 295 μg, up to about 300 μg, up to about 305 μg, up to about 310 μg, up to about 315 μg, up to about 320 μg, up to about 325 μg, up to about 330 μg, up to about 335 μg, up to about 340 μg, up to about 345 μg, up to about 350 μg, up to about 400 μg, up to about 450 μg, up to about 500 μg, up to about 540 μg, or up to about 650 μg every 4 weeks. Alternatively, or additionally, a dose or injection of 135 μg or 180 μg of deamidated Pro-hIFN A2b, Pro-hIFN A2b, and/or mixture thereof can be administered every 4 weeks.

In one embodiment, deamidated Pro-hIFN A2b, Pro-hIFN A2b, and/or mixture thereof can be administered or dosed for treating myelofibrosis of any subtype (e.g., idiopathic, primary, and/or early), polycythemia vera (high or low risk), essential thrombocythaemia, and/or chronic myeloid leukemia according to the formula 0.75-1.5 μg/kg/wk where the μg is amount of deamidated Pro-hIFN A2b, Pro-hIFN A2b, and/or mixture thereof, kg is a subject's weight and wk is week.

In some embodiments, an initial (starting) dose for treating myelofibrosis of any subtype (e.g., idiopathic, primary, and/or early), polycythemia vera (high or low risk), essential thrombocythaemia, and/or chronic myeloid leukemia can be about 250 to about 500 μg (e.g., about 250 μg, about 300 μg, about 350, about 400 μg, about 450 μg, or about 500 μg) of deamidated Pro-hIFN A2b, Pro-hIFN A2b, and/or mixture thereof administered to the subject. The initial dose/injection can be maintained or varied during the treatment period depending on patient's need and/or physician's recommendation.

In any of the methods or treatment periods and/or dosage described herein for deamidated Pro-hIFN A2b, Pro-hIFN A2b, and/or mixture thereof can be titrated. As non-limiting example, a subject can be treated for treating myelofibrosis of any subtype (e.g., idiopathic, primary, and/or early), polycythemia vera (high or low risk), essential thrombocythaemia, and/or chronic myeloid leukemia with a lower starting dose/injection (e.g., about 50 μg, about 100 μg, about 150 μg, about 200 μg, or about 250 to about 500 μg) of the deamidated Pro-hIFN A2b, Pro-hIFN A2b, and/or mixture thereof. If the subject responds well (e.g., lack of significant drug-related adverse events, significant self-reported discomfort, abnormal hematological responses, or other symptoms) after a time (e.g., between about 1 to 26 weeks, 1 to 52 weeks, or more than 52 weeks), the dose/injection given to the subject can be increased incrementally (e.g., by between about 50 to 250 μg, such as at about 50 μg, about 75 μg, about 100 μg, about 125 μg, about 150 μg, about 200 μg, about 250 μg or a combination thereof) every 2 to 16 weeks (e.g., every 1, 2, 3, 4, 5, 6, 7, 8, 9, 10, 11, 12, 13, 14, 15, or 16 weeks, or a combination thereof) until the dose reaches a target dose (e.g., at least about 400 μg, at least about 425 μg, at least about 450 μg, at least about 475 μg, at least about 500 μg, at least about 525 μg, at least about 550 μg, or at least about 650 μg). After that, a target dose can be maintained during the treatment period, increased, and/or decreased depend on subject's condition. The dose/injection can also be increased successively until the desired target dose is reached. For example, if the deamidated Pro-hIFN A2b, Pro-hIFN A2b, and/or mixture thereof can be administered once every 1, 2, 3, 4, 5, 6, 7, or 8 weeks, the dose/injection can be increased every 1, 2, 3, 4, 5, 6, 7 or 8 weeks, respectively. In some embodiments, a subject can be given a starting dose/injection of 250 μg (i.e., at week 0). If the subject responds well to the initial dose/injection, the dose/injection can be increased by about 100 to about 150 μg every 2 to 8 weeks until it reaches a target dose of about 500 μg, about 550 μg, and/or 600 μg. For example, a 250-350-500 μg dosing schedule can be implemented (i.e., about 250 μg at week 0, about 350 μg between about week 2 to 8, and about 500 μg at the third administration 2 to 8 weeks after the initial second dose, without other intervening doses). Alternative or additionally, a subject can be for treating myelofibrosis of any subtype (e.g., idiopathic, primary, and/or early), polycythemia vera (high or low risk), essential thrombocythaemia, and/or chronic myeloid leukemia by given a starting dose/injection of about 350 μg and a second dose of about 500 μg between about 2 to 16 weeks thereafter without an intervening dose (i.e., 350-500). Exemplary dosing schedules can be abbreviated, nonetheless, each number is approximated. For example, 250-350-500 includes 1st dose/injection administered at about 250 μg, 2nd dose/injection administered at about 350 μg, and 3rd dose/injection administered at about 500 μg. Other embodiments include, but are not limited to: 250-400-500, 250-400, 250-500, 250-400-500, 250-450, 250-350-400-500, 250-300-400-500, 250-350-450-500, 250-350-450, 250-250-350-500, 250-250-250-350-500, 250-350-350-500, 250-500-500-500 (and remain at 500 afterwards), 350-500, 350-400-500, 350-400-450-500, 350-450-500, 350-400, 350-450, 350-350-500, 350-350-350-500, 400-450-500, 400-500, 400-400-500, 450-500 μg. In some embodiments, the target dose and/or desirable effect is reached between about 1 to 13 weeks, about 1 to 26 weeks, 1 to 52 weeks, or more than 52 weeks from the initial administration. During the titration process, any dose, prior to reaching the target dose, may be maintained for a time period (e.g., between about 4 to 16 weeks) or a number of successive doses dose/injection (e.g., between 2 to 8 successive doses dose/injection, e.g., 250-350-350-500 μg) or reduced depending on the subject's response. In some embodiments, the target dose is reached within about 2 to 8 successive doses. In further embodiments, once the subject is clinically stable, the dose/injection can be maintained at a constant level for a given treatment period, which can be at least about 1, 2, 3, 4, 5, 6, 7, 8, 12, 14, 15, 16, 17, 18, 19, 20, 24, 28, 32, 36, 40, 44, 48, 52, 56, 60 weeks or longer.

In an aspect, an initial dose/injection or starting dose/injection of deamidated Pro-hIFN A2b, Pro-hIFN A2b, and/or mixture thereof refers to the first dose administered to a subject for treating myelofibrosis of any subtype (e.g., idiopathic, primary, and/or early), polycythemia vera (high or low risk), essential thrombocythaemia, and/or chronic myeloid leukemia during a treatment period (i.e., week 0), wherein, prior to the treatment period, the subject is interferon-treatment naïve or has not been administered the same active ingredient as deamidated Pro-hIFN A2b, Pro-hIFN A2b, and/or mixture thereof. A subject who is interferon-treatment naïve is a subject who has not been treated with any form of interferon, whether pegylated or non-pegylated.

In another embodiment, all of the above recited dosages for treating myelofibrosis of any subtype (e.g., idiopathic, primary, and/or early), polycythemia vera (high or low risk), essential thrombocythaemia, and/or chronic myeloid leukemia can be administered per dose, and/or per injection. Non-limiting example include administering about 250-350-500 μg of drug using about 50 μg per about 5 injections at about the same time and/or different times, so long as the subject receives a total of about 250 μg dose, or about 100 μg per injection about 5 times at about the same time and/or different times for a total of about 500 μg dose. The combination can be picked and choose depending on patient or subject convenience and/or medical need. In another non-limiting example, the entire about 250 μg can be all administered in a single injection (per injection) at a single time point for the desired 250 μg dose.

In an embodiment, any of the above-mentioned dosage, or dosage scheme can be use, and/or in a mix-and-match fashion for treating myelofibrosis of any subtype (e.g., idiopathic, primary, and/or early), polycythemia vera (high or low risk), essential thrombocythaemia, and/or chronic myeloid leukemia, depending on the subject's tolerance and a physician's assessment (e.g., based on number and/or types of AE). In another embodiment, all of the above recited dosages can be administered per dose, and/or per injection. Non-limiting example include administering about 250 μg, 350 μg of drug using about 600 μg per about 3 injections at about the same time and/or different times, so long as the subject receives a total of about 600 μg dose. The combination can be picked and choose depending on patient or subject convenience and/or medical need. In another non-limiting example, the entire about 600 μg can be all administered in a single injection (per injection) at a single time point.

In an aspect, deamidated Pro-hIFN A2b, Pro-hIFN A2b, and/or mixture thereof can be used for subcutaneous injection to a subject. Alternative, or additionally, deamidated Pro-hIFN A2b, Pro-hIFN A2b, and/or mixture thereof can be administered for treating myelofibrosis of any subtype (e.g., idiopathic, primary, and/or early), polycythemia vera (high or low risk), essential thrombocythaemia, and/or chronic myeloid leukemia via topical contact, intravenous, intraperitoneal, intramuscular, intralesional, intranasal or the implantation of a slow-release device e.g., a mini-osmotic pump, to a subject. Other administration is by any route including parenteral, and transmucosal (e.g., oral, nasal, vaginal, rectal, or transdermal). Parenteral administration includes, e.g., intravenous, intramuscular, intra-arteriole, intradermal, intraperitoneal, intraventricular, and/or intracranial. Moreover, where injection is to treat a tumor, e.g., induce apoptosis, administration may be directly to the tumor and/or into tissues surrounding the tumor. Other modes of delivery include, but are not limited to, the use of liposomal formulations, intravenous infusion, or transdermal patches.

Depending on the desired purpose(s), the pharmaceutical composition or medicament in accordance with the present disclosure can also be provided in other forms. For example, the pharmaceutical composition or medicament can be provided in a form for oral administration, intravenous injection (including drip infusion and bolus injection), intramuscular injection, subcutaneous injection, intraarterial injection, intraperitoneal injection, transdermal administration (such as a patch), or transmucosal administration (such as nasal spray, nasal drops, and suppository), but is not limited thereby. Depending on the form and purpose(s), a carrier can be chosen and used to provide the pharmaceutical composition or medicament, wherein the carriers are known in the art. Examples of the carrier include, but are not limited to, excipients, diluents, fillers, buffers, auxiliaries, stabilizers, absorption enhancers, disintegrating agents, hydrotropic agents, antioxidants, adhesives, binders, tackifiers, dispersants, suspending agents, lubricants, and hygroscopic agents.

In an embodiment, as a form for oral administration, the pharmaceutical composition or medicament in accordance with the present disclosure can be provided by any methods in any form for oral administration, wherein the liquid form suitable for oral administration includes syrups, an oral solution, a suspension, and an elixir, and the solid form suitable for oral administration includes a powder, a granule, a troche, a dragee, an enteric-coated tablet, a chewable tablet, an effervescent tablet, a film-coated tablet, a capsule, and a long-acting slow-release tablet. The pharmaceutical composition or medicament provided in accordance with the present disclosure can comprise any pharmaceutically acceptable carrier that will not adversely affect the desired effects of the active ingredient (i.e., antibodies of the present disclosure or antigen-binding fragment thereof). For example, the pharmaceutically acceptable carriers for the aforesaid liquid form include but are not limited to, water, saline, dextrose, glycerol, ethanol or its analogs, oil (e.g., olive oil, castor oil, cottonseed oil, peanut oil, corn oil, and germ oil), glycerol, polyethylene glycol, and combinations thereof, and the pharmaceutically acceptable carriers for the aforesaid solid form include, but are not limited to, cellulose, starch, kaolinite, bentonite, sodium citrate, gelatin, agar, carboxymethyl cellulose, gum arabic, tragacanth, seaweed gel, glyceryl monostearate, calcium stearate, colloidal silicon dioxide, and combinations thereof.

In another embodiment, as a form for transdermal administration, the pharmaceutical composition or medicament in accordance with the present disclosure can also comprise any pharmaceutically acceptable carrier that will not adversely affect the desired effects of the active ingredient (i.e., antibodies of the present disclosure or antigen-binding fragment thereof), such as water, mineral oil, propylene glycol, polyethylene oxide, liquid petrolatum, sorbitan monostearate, and polysorbate 60. The pharmaceutical composition or medicament can be provided by any suitable methods in any suitable form for transdermal administration, such as in the form of a patch (such as a microneedle patch), but is not limited thereby.

As for the form of injections or drips, the pharmaceutical composition or medicament can comprise one or more ingredient(s), such as an isotonic solution, a salt-buffered saline (e.g., phosphate-buffered saline or citrate-buffered saline), a hydrotropic agent, an emulsifier, a 5% sugar solution, and other carriers to provide the pharmaceutical composition or medicament as an intravenous infusion, an emulsified intravenous infusion, a powder for injection, a suspension for injection or a powder suspension for injection. Alternatively, the pharmaceutical composition or medicament can be prepared as a pre-injection solid. The desired injection is provided by dissolving the pre-injection solid in other solutions or suspensions or emulsifying it before being administered to a subject or subject in need.

In an embodiment, as a form for transmucosal administration, the pharmaceutical composition or medicament can comprise one or more ingredient(s), such as a penetrant, a surfactant, a viscosity regulator, a pH-adjusting agent, a preservative, a stabilizer, an osmo-regulator, and other carriers to provide the pharmaceutical composition or medicament as eye drops, an ointment, orally disintegrating tablets, a nasal spray, nasal drops, or a suppository.

Optionally, the pharmaceutical composition or medicament in accordance with the present disclosure can also comprise an amount of additives, such as a toner or a colorant for enhancing the visual perception of the pharmaceutical composition or medicament, and/or a buffer, a conservative, a preservative, an antibacterial agent, or an antifungal agent for improving the stability and storability of the pharmaceutical composition or medicament.

In certain embodiments, deamidated Pro-hIFN A2b, Pro-hIFN A2b, and/or mixture thereof is administered for treating myelofibrosis of any subtype (e.g., idiopathic, primary, and/or early), polycythemia vera (high or low risk), essential thrombocythaemia, and/or chronic myeloid leukemia once daily, about once weekly, about once every two weeks, about once every three weeks, about once every four weeks, about once every five weeks, about once every six weeks, or about once every three months. deamidated Pro-hIFN A2b, Pro-hIFN A2b, and/or mixture thereof can be co-administered within about twenty-four hours of a dose of chemotherapy and/or radiation therapy.

In certain embodiments, deamidated Pro-hIFN A2b, Pro-hIFN A2b, and/or mixture thereof is co-administered at least once about 3, once about 7, once about 10, once about 14, once about 17 or once about 21 days before a dose of chemotherapy and/or radiation therapy. In an embodiment, deamidated Pro-hIFN A2b, Pro-hIFN A2b, and/or mixture thereof can be co-administered to a patient immediately after, at about the same time, and/or any time during chemo and/or radiation therapy.

In one embodiment, deamidated Pro-hIFN A2b, Pro-hIFN A2b, and/or mixture thereof can be used to treat a subject with myelofibrosis of any subtype (e.g., idiopathic, primary, and/or early), polycythemia vera (high or low risk), essential thrombocythaemia, and/or chronic myeloid leukemia of any subtype.

In another embodiment, the deamidation can occur at position Asn46, Asn157, Gln159, or Gln102, and any combinations thereof on Pro-hIFN A2b.

Surprising Advantages and/or Improvement

Deamidation of asparagine and/or glutamine alters protein structures and affects the chemical and biological properties of proteins. Deamidation is a non-enzymatic reaction during which the covalent amide functional group is converted to a carboxylic acid. In proteins and peptides, deamidation can happen on asparagine (Asn) and/or glutamine (Gln) residues as a post-translational modification (PTM) to produce aspartic acid (Asp) and/or glutamic acid (Glu).

Under alkaline conditions, deamidation of Asn proceeds via a succinimide intermediate formed by the nucleophilic attack of the side chain carbonyl carbon by the backbone amine. The succinimide ring is then hydrolyzed at either the α- or β-carbonyl groups to produce isoAsp and Asp products. Under acidic conditions, Asn usually deamidates by direct hydrolysis via acid catalysis. However, the present method does not expose the protein in either acidic or alkaline stress; therefore, the identification of deamidation of Asn was surprising and unexpected.

Deamidation of Gln forms by direct hydrolysis at acidic pH and occurs via a glutarimide intermediate at alkaline pH. However, the present method does not expose the protein in alkaline stress; therefore, the identification of deamidation of Gln was surprising and unexpected.

Furthermore, in an embodiment, it is surprising and unexpected to find that the potency of deamidated Pro-IFN alfa-2b species retained full biological activity, which is in direct opposite of reported protein such as IFN-beta 1a or IFN-alpha 2a.

In addition, as compared to the previously reported method of making Pro-hIFN alpha 2b (see U.S. Pat. No. 8,106,160, the contents are all incorporated herein in their entirety), the present disclosure's method production of Pro-hIFN alpha 2b with deamidated Pro-hIFN A2b, and/or mixture thereof can be used as an indicator that any or all of the present disclosure's method of making was utilized and/or performed. For example, see FIG. 3 of U.S. Pat. No. 8,106,160, and/or the lack of known deamidated Pro-hIFN A2b, and/or mixture thereof in the art.

In addition, in another embodiment, not all batches of manufacturing include deamidated Pro-hIFN A2b.

Further, in an embodiment deamidated Pro-hIFN A2b, Pro-hIFN A2b, and mixture thereof does not always present in a given batch of manufacturing. In an occasion, deamidated Pro-hIFN A2b was not detected. In other embodiment, deamidated Pro-hIFN A2b Pro-hIFN A2b can be detected at 90%, 91%, 92%, 93%, 94%, 95%, 96%, 97%, 98%, 99%, 99.5%, 99.9% and/or 99.99% frequency over time.

In another embodiment, the deamidation can occur at position Asn46, Asn157, Gln159, and Gln102 or any combinations thereof on Pro-hIFN A2b

In one embodiment the deamidation can occur at one or more positions from Asn46, Asn157, Gln159, and Gln102, and any combinations thereof on Pro-hIFN A2b. For example, the one or more deaminated positions may be Asn46 and Asn157; Asn46 and Gln159; Asn46 and Gln102; Asn157 and Gln159; Asn157 and Gln102; Gln159 and Gln102; Asn46 Asn157 and Gln159; or Asn46 Asn157 and Gln102. In an embodiment, the mere existence of one or more of the deamidated species indicated in the present disclosure, shows that the present disclosure's method, in part or in whole was utilized.

Alternatively, or additionally, the deamidated Pro-hIFN A2b can include deamidation on one or more positions from Gln at position 21, Gln at position 41, Gln at position 47, Asn at position 66, Asn at position 94, and/or Gln at position 125 of SEQ ID No: 2 and the mere existence of one or more of the deamidated species indicated in the present paragraph, shows that the present disclosure's method, in part or in whole, was utilized.

In addition, as denoted above, Bandi et. al. reported four positions of N45, N65, N93 and N156 of IFN-α2a were mutated and replaced with D to obtain the deamidated IFN-α2a protein. It stated that as compared with wild type IFN-α2a, the potency (anti-proliferative and antiviral activities) of deamidated IFN-α2a was reduced by more than 120 times.

In an embodiment, the mere existence of one or more of the deamidated species indicated in the present disclosure, shows that the present disclosure's method, in part or in whole was utilized.

On the direct contrary, in the present disclosure, SPR data as shown in the below examples denote that the efficacy does not change by such a magnitude (e.g., see Table 4 below), which was surprising and unexpected, In addition to different deamidated position and/or amino acids.

### EXAMPLES

### Example 1

Preparation of Recombinant N-Terminal Modified hIFN-α2b

This method is not identical to known method of making Pro-hIFN A2b. Here, a nucleic acid encoding Pro-hIFN-α2b was obtained by amplifying hIFN-α2b using primers. Briefly, codens, ATG and CCG (encoding Met and Pro, respectively) were added to the 5′ of coden that encodes Cys of mature hIFN-α2b. This nucleic acid was cloned into expression vector pET-24a.

pET-24a vectors, carrying the Pro-hIFN-α2b genes, were then transformed into E. coli BLR-(DE 3)-RIL strain. E. coli clones expressing high levels of the protein were selected. The nascent protein expressed from the Pro-hIFN-α2b gene has a Met at its N-terminus (Met-Pro-Cys-). This Met residue was removed in E. coli via internal enzymatic digestion, resulting in a mature protein having an N-terminal Pro, which is linked to the Cys1 of hIFN-α2b (Pro-Cys-).

The E. coli clone which will express Pro-hIFN-α2b was cultured in a 1000 ml flask containing 250 ml SYN Broth medium (soytone, yeast extract, and NaCl) with karamycin (50 μg/mL) and chloramphenical (50 μg/mL) at 37° C., rotate at 200 rpm for about 16 hours overnight. 220 ml of the overnight culture were then transferred to a 5-liter jar fermenter containing 3 L define medium (10 g/L glucose, 0.7 g/L MgSO₄·7H₂O, 4 g/L (NH₄)₂HPO₄, 3 g/L KH₂PO₄, 6 g/L K₂HPO₄, 1.7 g/L citrate, 10 g/L yeast extract, 10 ml/L Trace Element, and 2 g/L isoleucine) with karamycin (25 μg/mL), chloramphenical (25 μg/mL), 0.4% glycerol, and 0.5% (v/v) trace elements (10 g/L of FeSO₄·7H₂O, 2.25 g/L of ZnSO₄·7H₂O, 1 g/L of CuSO₄·5H₂O, 0.5 g/L of MnSO₄·H₂O, 0.3 g/L of H₃BO₃, 2 g/L of CaCl₂·2H₂O, 0.1 g/L of (NH₄)₆Mo₇O₂₄, 0.84 g/L EDTA, and 50 ml/L HCl). The oxygen concentration in the medium was controlled at above 45% and its pH maintained at about 7.1 by adding a about 37% ammonia water whenever necessary. A feeding solution containing about 800 g/L of glucose and about 20 g/L of MgSO₄·7H₂O was prepared. When the dissolved oxygen rose to a value greater than the set point, an appropriate volume of the feeding solution was added to increase the glucose concentration in the culture medium. The expression of the Pro-hIFN-α2b gene was induced by IPTG at a final concentration of about 0.8 mM, then addition of feeding material (yeast extract and trace element). E. coli cells expressing these proteins were collected post five hours after IPTG induction.

In some runs, the fermentation step was utilized in accordance with the procedures and parameters as disclosed in U.S. Application publication number US20250101484A1. The mere existence of one or more of the deamidated Pro-hIFN A2b include positions from Gln at position 21, Gln at position 41, Gln at position 47, Asn at position 66, Asn at position 94, and/or Gln at position 125 of SEQ ID No: 2 indicated in the present paragraph, shows that the present disclosure's method (e.g., methods in US20250101484A1), in part or in whole, was utilized. In addition, The mere existence of one or more of the deamidated Pro-hIFN A2b include positions from Asn46, Asn157, Gln102, Gln159, Gln at position 21, Gln at position 41, Gln at position 47, Asn at position 66, Asn at position 94, and/or Gln at position 125 of SEQ ID No: 2 indicated in the present paragraph, shows that the present disclosure's method (e.g., methods in US20250101484A1), in part or in whole, was utilized.

The collected E. coli cells were resuspended in TEN buffer (50 mM Tris-HCl, pH 7.0; 1 mM EDTA, and 100 mM NaCl) in a ratio of about 1:10 (wet weight g/mL), disrupted by a homogenizer, and then centrifuged at 10,000 rpm for 20 min. The pellet containing inclusion bodies (IBs) was washed twice with TEN buffer and centrifuged as described above, suspended in a ratio of 1 ml solution:2.5 g pellet wet weight g/mL of a about 4 M guanidium HCl (GnHCl) aqueous solution, and then centrifuged at 5,000 rpm for 15 min. The IBs, containing recombinant hIFN-α2b, were then solubilized in 50 mL of 6 M GuHCl with 5 mM DTT, which was then stirred at room temperature for 1.5 hr followed by centrifugation at 20,000 rpm for 20 min at 25° C. The supernatant was collected. In this process, the recombinant Pro-hIFN A2b protein was denatured. The mere existence of one or more of the deamidated species Pro-hIFN A2b include positions from Gln at position 21, Gln at position 41, Gln at position 47, Asn at position 66, Asn at position 94, and/or Gln at position 125 of SEQ ID No: 2 indicated in the present paragraph, shows that the present paragraph's method, in part or in whole, was utilized. In addition, The mere existence of one or more of the deamidated Pro-hIFN A2b include positions from Asn46, Asn157, Gln102, Gln159, Gln at position 21, Gln at position 41, Gln at position 47, Asn at position 66, Asn at position 94, and/or Gln at position 125 of SEQ ID No: 2 indicated in the present paragraph, shows that the present paragraph's method, in part or in whole, was utilized.

### Example 2

### Gel Electrophoresis

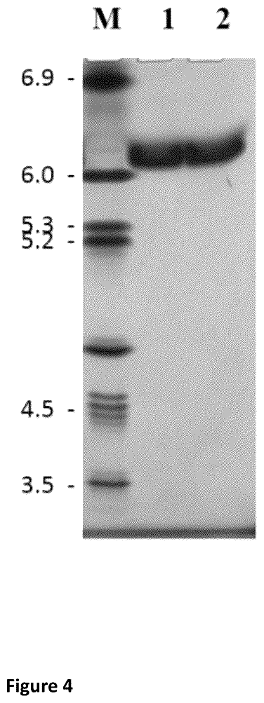

The protein concentration of the crude lysate was determined by BCA protein assay, and the Pro-hIFN A2b was determined by isoelectric focusing gel electrophoresis. The charge distribution of Pro-IFN alfa-2b was also evaluated by isoelectric focusing (IEF) gel electrophoresis. The results are illustrated in FIG. 4, with duplicated lanes each loaded with about 15 μg with a marker line loaded with about 10 uL on the very left labelled as “M”. FIG. 4 shows an IEF profile that gives major band with the isoelectric point (pI) of 6.1 for Pro-IFN alfa-2b. Results are consistent with the detection of an IEF main band at pI 6.1±0.1. The mere existence of one or more of the deamidated species Pro-hIFN A2b include positions from Gln at position 21, Gln at position 41, Gln at position 47, Asn at position 66, Asn at position 94, and/or Gln at position 125 of SEQ ID No: 2 indicated in the present paragraph, shows that the present paragraph's method, in part or in whole, was utilized. In addition, The mere existence of one or more of the deamidated Pro-hIFN A2b include positions from Asn46, Asn157, Gln102, Gln 159, Gln at position 21, Gln at position 41, Gln at position 47, Asn at position 66, Asn at position 94, and/or Gln at position 125 of SEQ ID No: 2 indicated in the present paragraph, shows that the present paragraph's method, in part or in whole, was utilized.

### Example 3

### Refolding of Pro-hIFN Alpha 2b.

The above-described IBs were mixed with 1.5 L freshly prepared refolding buffer (100 mM Tris-HCl (pH 7.0), 0.5 M L-Arginine, 2 mM EDTA). The reaction mixture thus formed was incubated for 24˜36 hr. without stirring at room temperature to allow refolding of the recombinant Pro-hIFN alpha 2b. The mere existence of one or more of the deamidated species Pro-hIFN A2b include positions from Gln at position 21, Gln at position 41, Gln at position 47, Asn at position 66, Asn at position 94, and/or Gln at position 125 of SEQ ID No: 2 indicated in the present paragraph, shows that the present paragraph's method, in part or in whole, was utilized. In addition, The mere existence of one or more of the deamidated Pro-hIFN A2b include positions from Asn46, Asn157, Gln102, Gln159, Gln at position 21, Gln at position 41, Gln at position 47, Asn at position 66, Asn at position 94, and/or Gln at position 125 of SEQ ID No: 2 indicated in the present paragraph, shows that the present paragraph's method, in part or in whole, was utilized.

### Example 4

### Ultrafiltration/Diafiltration

This method is not identical compared to known method of making Pro-hIFN A2b. Here the refolded mixture was centrifuged under 10,000±1,000 rpm at room temperature. The collected supernatant was filtered through 0.2 μm filters and clarified solution was obtained. The filtrate was then concentrated 20 folds by a tangential Ultrafiltration system and diafiltered in constant-volume-mode with about 7 volumes of Q-A equilibrium buffer (pH 7.0±0.1, conductivity<2 mS/cm). Finally, the retentate suspension was harvested. The suspension was then centrifuged at 9,000 rpm, 25 min at 4° C. The collected supernatant was then pumped through a Sartorius 0.2 μm filter. A volume of 10 mL sample was taken out for protein concentration analysis by the BCA method. The filtrate was obtained and stored in refrigerator overnight at 4° C. The mere existence of one or more of the deamidated species Pro-hIFN A2b include positions from Gln at position 21, Gln at position 41, Gln at position 47, Asn at position 66, Asn at position 94, and/or Gln at position 125 of SEQ ID No: 2 indicated in the present paragraph, shows that the present paragraph's method, in part or in whole, was utilized. In addition, The mere existence of one or more of the deamidated Pro-hIFN A2b include positions from Asn46, Asn157, Gln102, Gln 159, Gln at position 21, Gln at position 41, Gln at position 47, Asn at position 66, Asn at position 94, and/or Gln at position 125 of SEQ ID No: 2 indicated in the present paragraph, shows that the present paragraph's method, in part or in whole, was utilized.

### Example 5

### Ammonium Acetate/Sulfate Precipitation (AAP/ASP)

This example is another illustration of departure of known method of making Pro-hIFN alpha 2b. Here a buffer for ammonium acetate precipitation was prepared to contain about 48 g/L ammonium acetate (about 0.6 M NH₄OAc) and about 180 g/L sodium chloride at about pH 2.8. The precipitation was performed by adding 40 ml AAP buffer to Pro-hIFN alpha 2b in glass bottle with stirring. The solution was then placed in 40° C. water bath and incubated for about 2 h. After incubation, the mixture was then clarified by centrifugation at about 9,000 rpm for about 20 min at 4° C. The pooled supernatant was adjusted to pH about 4.5. After the pooled supernatant was filtered by 0.2 μm filtration membrane, the filtrate was stored at 4° C. overnight. The mere existence of one or more of the deamidated species Pro-hIFN A2b include positions from Gln at position 21, Gln at position 41, Gln at position 47, Asn at position 66, Asn at position 94, and/or Gln at position 125 of SEQ ID No: 2 indicated in the present paragraph, shows that the present paragraph's method, in part or in whole, was utilized. In addition, The mere existence of one or more of the deamidated Pro-hIFN A2b include positions from Asn46, Asn157, Gln102, Gln 159, Gln at position 21, Gln at position 41, Gln at position 47, Asn at position 66, Asn at position 94, and/or Gln at position 125 of SEQ ID No: 2 indicated in the present paragraph, shows that the present paragraph's method, in part or in whole, was utilized.

### Example 6

### Purification (Anionic Exchange, AIX)

AIX column flow rate was at about 35 ml/min. there are two washing steps in before elution. The elution step were performed at 15 about CV of elution buffer containing about 80 mM NaCl was used. The elution of Pro-hIFN alpha 2b, deamidated Pro-hIFN alpha 2b or mixture thereof were all monitored by 280 nm UV detection. The wash condition is set at between 15-25 mM of wash salt solution, and the elution buffer has the salt concentration of between 75-85 mM. pH is maintained at about 7. The mere existence of one or more of the deamidated species Pro-hIFN A2b include positions from Gln at position 21, Gln at position 41, Gln at position 47, Asn at position 66, Asn at position 94, and/or Gln at position 125 of SEQ ID No: 2 indicated in the present paragraph, shows that the present paragraph's method, in part or in whole, was utilized. In addition, The mere existence of one or more of the deamidated Pro-hIFN A2b include positions from Asn46, Asn157, Gln102, Gln159, Gln at position 21, Gln at position 41, Gln at position 47, Asn at position 66, Asn at position 94, and/or Gln at position 125 of SEQ ID No: 2 indicated in the present paragraph, shows that the present paragraph's method, in part or in whole, was utilized.

### Example 7

### N-Terminal Sequencing and Peptide Mapping of Pro-IFN-α2b

The tested sample was subjected to Edman degradation chemistry that uses phenylisothiocyanate to react with the uncharged N-terminal amino group on the protein sample. The resulting phenylthiohydantoin (PTH)-amino acid derivative is stable and can be identified by RP-HPLC.

Peptide mapping was employed to analyze the sequence of Pro-IFN alfa-2b. Peptide maps were generated by the RP-HPLC separation of peptide mixtures resulting either from trypsin and endoproteinase Lys-C dual-digestion or from endoproteinase Glu-C digestion of a sample. After the digestion, the proteolytic reaction was quenched by the addition of trifluoroacetic acid (TFA) to the final concentration of 0.4% (v/v). The resulting peptide mixtures were separated using RP-HPLC with TFA/acetonitrNle (ACN) gradient to generate a non-reduced map. To obtain a reduced map, peptide mixtures were reduced with tris (2-carboxyethyl) phosphine hydrochloride (TCEP-HCl) after the reaction is quenched and then subjected to RP-HPLC. The eluted peptide peaks were monitored by UV detection and subsequently analyzed by on-line LC-MS/MS for detection of peptide masses and sequence of selected peptides.

### Trypsin/Lys-C Peptide Mapping

The protein sample was digested with trypsin at 37° C. for 16 hr (trypsin/protein sample=1/80, w/w) in 50 mM sodium phosphate buffer, pH 6.8. Next, an aliquot of 10 milli-amidase unit (mAU) of Lysyl Endopeptidase (Lys-C) was added to the tryptic digested peptide mixture at 37° C. for 4 hr. The reaction was quenched by adding trifluoroacetic acid (TFA) to the final concentration of 0.4% (v/v). The mixture was subsequently reduced by 4 mM of tris (2-carboxyethyl) phosphine hydrochloride (TCEP-HCl) at 37° C. for 1.5 hr.

Table 1 summarizes the identification of all peptides obtained from the reduced trypsin/Lys-C peptide map. All expected peaks with respective sequence assignment and measurement of accurate masses are observed except for short peptide fragments that are not recoverable on the maps. These fragments contain only a single residue or dipeptides, such as T2, T4, T6, T12, T15, T16, T20, and T21; and some of them can be identified in partially digested long peptides such as T2-3, T6-7, T12-13 and T16-17.

TABLE 1

| | | | Monoisotopic | |
|---|---|---|---|---|
| Peptide | Residue | Sequence | Mass (cal.) | observed |
| T1 | 1-13 | PCDLPQT | 1409.67 | 1409.68 |
| | | HSLGSR | | |
| T1a | 1-8 | PCDLPQTH | 909.40 | 909.41 |
| T2 | 14 | R | 174.11 | ND |
| T3 | 15-23 | TLMLL | 1075.59 | 1075.60 |
| | | AQMR | | |
| T4 | 24 | R | 174.11 | ND |
| T5 | 25-32 | ISLFSCLK | 909.50 | 909.51 |
| T? | 33-34 | DR | 289.14 | ND |
| T7 | 35-50 | HDFGFPQ | 1953.86 | 1953.87 |
| | | EEFGNQF | | |
| | | QK | | |
| T6-7 | 33-50 | DRHDFG | 2224.99 | 2225.00 |
| | | FPQEEF | | |
| | | GNQFQK | | |
| T8 | 51-71 | AETIPV | 2458.29 | 2458.31 |
| | | LHEMIQ | | |
| | | QIFNLF | | |
| | | STK | | |
| T9 | 72-84 | DSSAAWD | 1449.66 | 1449.67 |
| | | ETLLDK | | |
| T10 | 85-113 | FYTELYQ | 3301.63 | 3301.65 |
| | | QLNDLEA | | |
| | | CVIQGVG | | |
| | | VTETPLM | | |
| | | K | | |
| T11 | 114-121 | EDSILAVR | 901.49 | 901.50 |
| T12 | 122 | K | 146.11 | ND |
| T13 | 123-126 | YFQR | 612.30 | 612.31 |
| T12-13 | 122-126 | KYFQR | 740.40 | 740.41 |
| T14 | 127-132 | ITLYLK | 749.47 | 749.48 |
| T15 | 133-134 | EK | 275.15 | ND |
| T16 | 135 | K | 146.11 | ND |
| T17 | 136-145 | YSPCA | 1208.57 | 1208.58 |
| | | WEVVR | | |
| T16-17 | 134-145 | KYSPCA | 1336.66 | 1336.67 |
| | | WEVVK | | |
| T18 | 146-150 | AEIMR | 618.32 | 618.32 |
| T19 | 151-163 | SFSLST | 1480.75 | 1480.76 |
| | | NLQESLR | | |
| T20 | 164-165 | SK | 233.14 | ND |
| T21 | 166 | E | 147.05 | ND |

The mere existence of one or more of the deamidated species Pro-hIFN A2b include positions from Gln at position 21, Gln at position 41, Gln at position 47, Asn at position 66, Asn at position 94, and/or Gln at position 125 of SEQ ID No: 2 indicated in the present paragraph, shows that the present paragraph's method, in part or in whole, was utilized. In addition, The mere existence of one or more of the deamidated Pro-hIFN A2b include positions from Asn46, Asn157, Gln102, Gln159, Gln at position 21, Gln at position 41, Gln at position 47, Asn at position 66, Asn at position 94, and/or Gln at position 125 of SEQ ID No: 2 indicated in the present paragraph, shows that the present paragraph's method, in part or in whole, was utilized.

### Example 8

Unexpected Detection and Analysis of Pro-IFN-α2b with Deamidation.

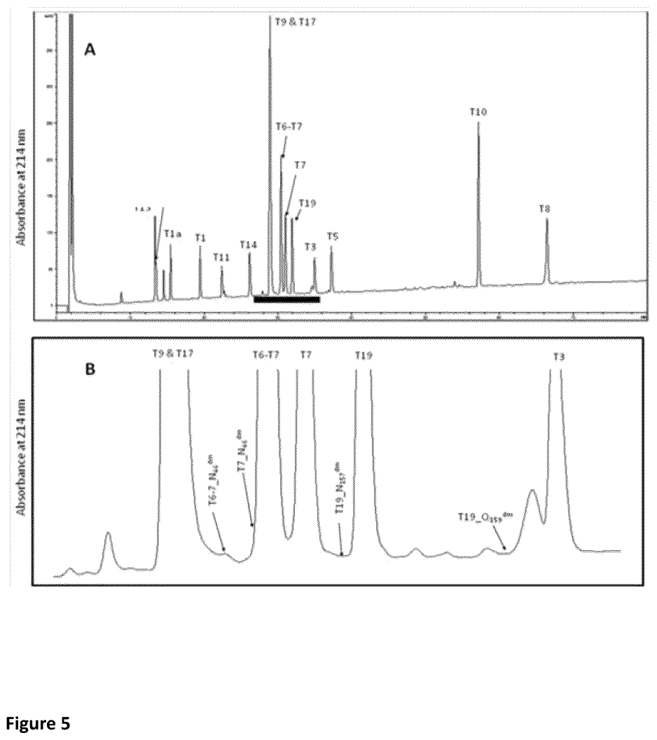

Although peptide mapping showed correct amino acid sequences, the same was further analyzed with IEX-HPLC of Pro-IFN alfa-2b form and charged isoforms. Table 2 summarizes the content of the deamination positions. The content of deamidated species in Pro-IFN alfa-2b IEX-HPLC identified deamidated peptides with a mass increase of approximately 1 Da. Deamidation was observed in Pro-IFN alfa-2b by tryptic peptide mapping as shown in FIG. 5. Quantification is based on MS extracted ion chromatogram (XIC) for deamidated peptide identified in the map and results are listed in Table 2 below.

In one run, the deamidation specie was detected at one or more positions from Asn46, Asn157, Gln159, and Gln102, and/or any combinations thereof on Pro-hIFN A2b.

In one embodiment, the deamidation species were detected at position Asn46 and Asn157 of Pro-hIFN A2b.

In one embodiment, the deamidation species were detected at position Asn46 and Gln159 of Pro-hIFN A2b.

In one embodiment, the deamidation species were detected at position Asn46 and Gln102 of Pro-hIFN A2b.

In one embodiment, the deamidation species were detected at position Asn157 and Gln159 of Pro-hIFN A2b.

In one embodiment, the deamidation species were detected at position Asn157 and Gln102 of Pro-hIFN A2b.

In one embodiment, the deamidation species were detected at position Gln159 and Gln102 of Pro-hIFN A2b.

In one embodiment, the deamidation species were detected at position Asn46 Asn157 and Gln159 of Pro-hIFN A2b.

In one embodiment, the deamidation species were detected at position Asn46 Asn157 and Gln102 of Pro-hIFN A2b.

The mere existence of one or more of the deamidated species Pro-hIFN A2b include positions from Gln at position 21, Gln at position 41, Gln at position 47, Asn at position 66, Asn at position 94, and/or Gln at position 125 of SEQ ID No: 2 indicated in the present paragraph, shows that the present paragraph's method, in part or in whole, was utilized. In addition, The mere existence of one or more of the deamidated Pro-hIFN A2b include positions from Asn46, Asn157, Gln102, Gln159, Gln at position 21, Gln at position 41, Gln at position 47, Asn at position 66, Asn at position 94, and/or Gln at position 125 of SEQ ID No: 2 indicated in the present paragraph, shows that the present paragraph's method, in part or in whole, was utilized. Table 2

| Deamidated | | % |
|---|---|---|
| Peptide | Deamidation Site | Deamidation*(n = 2) |
| T6-T7 & T7 | Asn (N) 46 | 0.8 |
| T10 | Gln (Q) 102 | 0.1 |
| T19 | Asn (N) 157 | 0.2 |
| T19 | Gln (Q) 159 | 0.5 |

CPE (Cytopathic effect) assay showed that the deamidated Pro-IFN-α2b with one or more deaminated Pro-IFN alfa-2b has no potency change.

### Example 9

### Multiple Runs

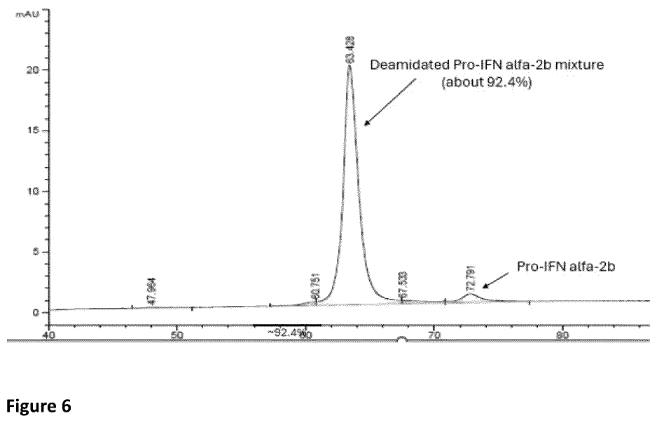

Multiple runs of Pro-IFN alfa-2b were synthesized again in accordance with the above examples, some with pH adjustment. Using the same methods and/or peptide mapping and chromatography method as denoted in Examples, the resulting deamidation showed at least about 15% of deamidated species mixture. Further enrichment was achieved by IEX-HPLC to reach a relative concentration of at least about 92.4% of deamidated species mixture as shown in FIG. 6. Additional analysis of the deamidated mixture showed more deamidation species and relative amount as denoted in Table 3.

TABLE 3
Modification Site

Q21 + Deamidation
Q41 + Deamidation
N46 + Deamidation
Q47 + Deamidation
N66 + Deamidation
N94 + Deamidation
Q102 + Deamidation
Q125 + Deamidation
N157 + Deamidation

In one of the enrichments run, the total amount of deamidated can reach at least about 15% and in another enrichment example, as high as 92.5%. Further examination of the deamidated positions showed that not all positions were produced all the time. The mere existence of one or more of the deamidated species indicated in the present disclosure, showed that the present disclosure's method, in part or in whole was utilized.

The mere existence of one or more of the deamidated species Pro-hIFN A2b include positions from Gln at position 21, Gln at position 41, Gln at position 47, Asn at position 66, Asn at position 94, and/or Gln at position 125 of SEQ ID No: 2 indicated in the present paragraph, shows that the present paragraph's method, in part or in whole, was utilized. In addition, The mere existence of one or more of the deamidated Pro-hIFN A2b include positions from Asn46, Asn157, Gln102, Gln159, Gln at position 21, Gln at position 41, Gln at position 47, Asn at position 66, Asn at position 94, and/or Gln at position 125 of SEQ ID No: 2 indicated in the present paragraph's method, shows that the present disclosure's, in part or in whole, was utilized.

### Example 10

### Additional Runs

In one of the multiple runs, the fermentation steps were carried out as disclosed in U.S. Patent Application Publication Number US20250101484A1, in particular, the fermentation was carrying out as: seeding (time S₀) of a culture of prokaryote host cell engineered to inducibly express a recombinant protein and transferring the culture into one or more fermenters each containing a culture medium; measuring a dissolved oxygen (DO) level, an agitation rate, and a pH in the one or more fermenters; supplying a carbon source feeding solution to any of the one or more fermenters individually, whenever condition(s) (i) and/or (ii) are met: (i) said DO level exceeds above about 35% to 45% and said agitation rate exceeds about 300 to 1,000 rpm; (ii) said agitation rate exceeds about 400 to 700 rpm and said pH exceeds about 7.0 to 7.4; and supplying a nitrogen source to any of the one or more fermenters in which induction of expression of the recombinant protein has been initiated, individually, at about I₀ (time at initiation of induction) or I₁ or both; optionally, supplying a nitrogen source to any of the one or more fermenters, individually, at one or more time points from about S₅, S₆, S₇, S₈, S₉ and S₁₀. The mere existence of one or more of the deamidated species indicated in the present disclosure, shows that the present disclosure's method, in part or in whole was utilized.

In another one of the multiple runs, deamidated species were also detected with Gln at position 21 was at least about 0.76%, the percentage of deamidated species for Gln at position 41 was at about least 0.02%, the percentage of deamidated species for Asn at position 46 was at least about 92%, the percentage of deamidated species for Gln at position 47 was at least about 0.15%, the percentage of deamidated species for Asn at position 66 is at least about 0.08%, the percentage of deamidated species for Asn at position 94 was at least about 0.11%, the percentage of deamidated species for Gln at position 102 was at least about 0.32%, the percentage of deamidated species for Gln at position 125 was at least about 0.10%, and/or the percentage of deamidated species for Asn at position 157 was at least about 2.59%. The mere existence of one or more of the deamidated species indicated in the present disclosure, showed that the present disclosure's method, in part or in whole was utilized.

In another run, deamidated species were detected of deamidated Pro-hIFN, where the percentage of deamidated species for Gln at position 21 was at least about 0.12%, the percentage of deamidated species for Gln at position 41 was at about least 0.29%, the percentage of deamidated species for Asn at position 46 was at least about 61.64%, the percentage of deamidated species for Gln at position 47 was at least about 0.09%, the percentage of deamidated species for Asn at position 66 is was least about 0.06%, the percentage of deamidated species for Asn at position 94 was at least about 0.11%, the percentage of deamidated species for Gln at position 102 was at least about 2.37%, the percentage of deamidated species for Gln at position 125 was at least about 0.98%, and/or the percentage of deamidated species for Asn at position 157 was at least about 17.87%. The mere existence of one or more of the deamidated species Pro-hIFN A2b include positions from Gln at position 21, Gln at position 41, Gln at position 47, Asn at position 66, Asn at position 94, and/or Gln at position 125 of SEQ ID No: 2 indicated in the present paragraph, shows that the present disclosure's method above, in part or in whole, was utilized. In addition, The mere existence of one or more of the deamidated Pro-hIFN A2b include positions from Asn46, Asn157, Gln102, Gln159, Gln at position 21, Gln at position 41, Gln at position 47, Asn at position 66, Asn at position 94, and/or Gln at position 125 of SEQ ID No: 2 indicated in the present paragraph, shows that the present disclosure's method above, in part or in whole, was utilized.

Yet in run, the amount of deamidated Pro-hIFN can include one or more of the following, where the percentage of deamidated species for Gln at position was is at least about 0.10%, the percentage of deamidated species for Gln at position 41 was at about least 0.20%, the percentage of deamidated species for Asn at position 46 was at least about 15.71%, the percentage of deamidated species for Gln at position 47 was at least about 0.08%, the percentage of deamidated species for Asn at position 66 was at least about 0.07%, the percentage of deamidated species for Asn at position 94 was at least about 0.14%, the percentage of deamidated species for Gln at position 102 was at least about 0.10%, the percentage of deamidated species for Gln at position 125 was at least about 0.06%, and/or the percentage of deamidated species for Asn at position 157 was at least about 0.54%. The mere existence of one or more of the deamidated species indicated in the present disclosure, showed that the present disclosure's method, in part or in whole was utilized.

In yet another run, the percentage of deamidated species for Gln at position 21 was at least about 0.1%, the percentage of deamidated species for Asn at position 46 was at least about 92%, the percentage of deamidated species for Asn at position 157 was at least about 2.5%. The mere existence of one or more of the deamidated species indicated in the present disclosure, showed that any step or steps of the present disclosure's method, in part or in whole was utilized.

In another run, the deamidated species, after analysis and quantification, showed that the quantification amount of the deamidated Pro-hIFN alpha 2b at Gln 21, Gln 41 and Gln 47 ratio was about 1:2:0.8. In the next run, the quantification amount of the deamidated Pro-hIFN alpha 2b at Gln 21, Gln 41 and Gln 47 ratio was about 2:4.8:1.5. In another run, the quantification amount of the deamidated Pro-hIFN alpha at 2b Gln 21, Gln 41 and Gln 47 ratio was about 38:1:8. The mere existence of one or more of the deamidated species indicated in the present disclosure, or in any of the quantity/ratio stated in the present disclosure, showed that any step or steps of the present disclosure's method, in part or in whole was utilized.

In a further run, an analysis of the deamidated Pro-hIFN alpha 2b at Asn 66, Asn 46 and Gln 125 ratio was about 0.8:1.4:3.8. In a run, the deamidated Pro-hIFN alpha 2b at Asn 66, Asn 46 and Gln 125 ratio was about 1:1.8:16. Yet in another run, an analysis of the deamidated Pro-hIFN alpha 2b at Asn 66, Asn 46 and Gln 125 ratio was about 4:5.5:5. The mere existence of one or more of the deamidated species indicated in the present disclosure, or in any of the quantity/ratio stated in the present disclosure, showed that any step or steps of the present disclosure's method, in part or in whole was utilized.

Yet in another run, the quantification amount of the deamidated Pro-hIFN alpha 2b at Gln 21, Gln 41, Gln 47, Asn 66, Asn 46 and Gln 125 ratio was about 1:2:0.8:0.8:1.4:3.8. In another run, the quantification amount of the deamidated Pro-hIFN alpha 2b at Gln 21, Gln 41, Gln 47, Asn 66, Asn 46 and Gln 125 ratio was about 2:4.8:1.5:1.:1.8:16. In another run, the quantification amount of the deamidated Pro-hIFN alpha 2b at Gln 21, Gln 41, Gln 47, Asn 66, Asn 46 and Gln 125 ratio was about 2:4.8:1.5:1.:1.8:16. In an run, the quantification amount of the deamidated Pro-hIFN alpha 2b at Gln 21, Gln 41, Gln 47, Asn 66, Asn 46 and Gln 125 ratio was about 38:1:8:4:5.5:5. The mere existence of one or more of the deamidated species indicated in the present disclosure, or in any of the quantity/ratio stated in the present disclosure, showed that any step or steps of the present disclosure's method, in part or in whole was utilized.

In one run, no deamidated species are detected.

### Example 11

### Multi-Cycle Kinetic Analysis Using SPR System

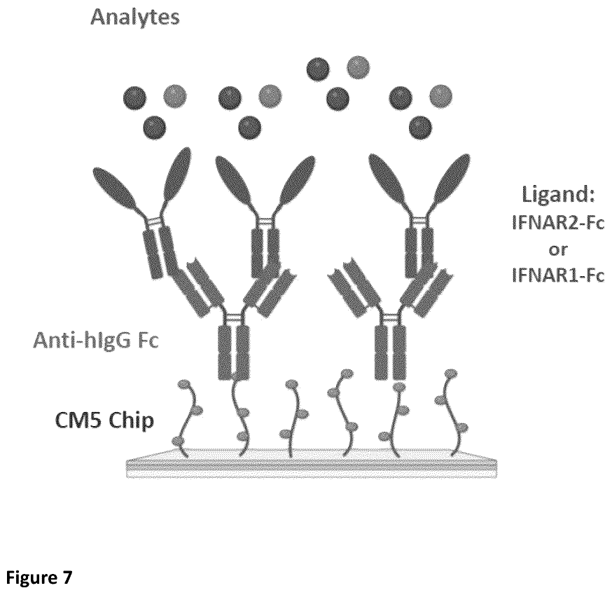

In this example, the ligand human IFNAR1-Fc/human IFNAR2-Fc was captured on the surface of a Sensor Chip Protein A. The multi-cycle kinetic analysis of Pro-IFN alfa-2b/deaminated Pro-IFN alfa-2b mixture to human IFNAR1-Fc/human IFNAR2-Fc was performed by using Biacore T200. The basic design is illustrated in FIG. 7.

Briefly. ligand (human IFNAR1-Fc/human IFNAR2-Fc) were used to capture the surface of a Sensor Chip Protein A. Human IFNAR1-Fc/human IFNAR2-Fc was first diluted with 1×HBS-EP+ to obtain a final concentration of 8 μg/ml and captured by flow cell 4 of a Sensor Chip Protein A. The chip contains MabSelect SuRe ligand on the surface, allowing orientation-specific binding of the Fc region of an antibody

The assay was performed by using the Kinetic/Affinity wizard. The flow path was 4-3, as the ligand was injected and captured in flow cell 4, and flow cell 3 acted as a reference. A series of concentrations of Pro-IFN alfa-2b/deamidated Pro-IFN alfa-2b mixture (also known as “analyte”). Batches with various relative concentrations of total deamidated species such as 15% (batch 1 below) and/or 92% (batch 2 below) were produced, then injected over the reference and the ligand surfaces consecutively as the association phase, with short dissociation phases in between by injecting running buffer. Then, regeneration solution was injected as the regeneration phase. All the procedures were conducted at 25° C.

All resulting data were analyzed and fitted to a steady-state affinity model or a kinetic 1:1 binding model using Biacore T200 Evaluation Software, version 3. Flow cell 3 and blank injection of running buffer were used as double references for subtraction. A summary of the results is listed in Table 4. In an embodiment, the mere existence of one or more of the deamidated species indicated in the present disclosure, shows that the present disclosure's method, in part or in whole was utilized.

TABLE 4

| | | | | Pro-IFN-α2b | Analyte Batch 1 | Analyte Batch 2 |
|---|---|---|---|---|---|---|
| Ligand | human | Steady-state | KD (M) | 3.67E⁻⁰⁶ | 2.75E⁻⁰⁶ | 2.06E⁻⁰⁶ |
| | IFNAR1-Fc | affinity | | | | |
| | | model | | | | |
| | | analysis | | | | |
| | human | Kinetic 1:1 | KD (M) | 1.44E⁻⁰⁹ | 1.64E⁻⁰⁹ | 2.29E⁻⁰⁹ |
| | IFNAR2-Fc | binding | | | | |
| | | model | | | | |
| | | analysis | | | | |

In the SPR analysis, the ligand human IFNAR1-Fc/human IFNAR2-Fc was captured on the surface of a Sensor Chip Protein A. For IFNAR1, the KD values ranged from 2.06 to 3.67 μM, while for IFNAR2, the analytes exhibited a higher binding affinity, with KD values ranging from 1.44 to 2.29 nM. The two deamidated analytes showed a binding affinity to human IFNAR1-Fc and human IFNAR2-Fc comparable to that of the reference standard. In sum, this example showed that deamidation of one or more, or all of the deamidated species in the present disclosure did not impact biological function such as potency or anti-viral activity, which in the direct contrary as reported in Bandi et al.

It is contemplated that any embodiment discussed in this specification can be implemented with respect to any method, kit, reagent, or composition of the disclosure, and vice versa. Furthermore, compositions of the disclosure can be used to achieve methods of the disclosure.

It will be understood that particular embodiments described herein are shown by way of illustration and not as limitations of the disclosure. The principal features of this disclosure can be employed in various embodiments without departing from the scope of the disclosure. Those skilled in the art will recognize, or be able to ascertain using no more than routine experimentation, numerous equivalents to the specific procedures described herein. Such equivalents are considered to be within the scope of this disclosure and are covered by the claims.

All publications and patent applications mentioned in the specification are indicative of the level of skill of those skilled in the art to which this disclosure pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and/or methods and in the steps or in the sequence of steps of the method described herein without departing from the concept, spirit and scope of the disclosure. All such similar substitutes and modifications apparent to those skilled in the art are deemed to be within the spirit, scope and concept of the disclosure as defined by the appended claims.

1. A composition comprising deamidated Pro-hIFN A2b, Pro-hIFN A2b, and/or mixture thereof.

2. The composition according to claim 1, wherein said deamidated Pro-hIFN A2b comprising deamidation at one or more positions from Asn at position 46, Gln at position 159, Asn at position 157, and/or Gln at position 102.

3. The composition according to claim 1, wherein said deamidated Pro-hIFN A2b comprising deamidation at one or more positions from Gln at position 21, Gln at position 41, Gln at position 47, Asn at position 46, Asn at position 66, Asn at position 94, Gln at position 125, Gln at position 159, Asn at position 157, and/or Gln at position 102.

4. The composition according to claim 1, wherein said deamidated Pro-hIFN A2b comprising deamidation at one or more positions from Gln at position 21, Gln at position 41, Gln at position 47, Asn at position 66, Asn at position 94, and/or Gln at position 125.

5. A pharmaceutical formulation comprising the composition according to claim 1; and
one or more pharmaceutically acceptable carrier.

6. A method for treating a subject or subject in need who has myelofibrosis, polycythemia vera, essential thrombocythaemia, and/or chronic myeloid leukemia comprising
administrating to the subject a therapeutically effective amount of the composition according to claim 1.

7. The method according to claim 6, wherein the myelofibrosis comprises idiopathic, primary, and/or early myelofibrosis.

8. The method according to claim 6, wherein the polycythemia vera comprises high and/or low risk polycythemia vera.

9. A method for producing the composition according to claim 1, the method comprising:
cloning a vector using DNA encoding the amino acid sequence of Pro-hIFN alpha 2b;
transfecting said vector into a host cell
fermenting said host cell at a condition including at about 37° C. with a dissolved oxygen at about 40%, wherein the fermentation pH is optionally adjusted to an alkaline condition;
inducing Pro-hIFN expression with IPTG at a concentration of about 0.8 mM;
removing the initial methionine from said Pro-hIFN;
refolding said Pro-hIFN protein;
performing ultrafiltration on said Pro-hIFN protein;
performing amino acetate precipitation on said Pro-hIFN protein; and
purifying said Pro-hIFN protein.

10. The method for producing the composition according to claim 9, wherein said fermenting comprises the steps of:
carrying out seeding (time S₀) of a culture of prokaryote host cell engineered to inducibly express a recombinant protein and transferring the culture into one or more fermenters each containing a culture medium;
measuring said dissolved oxygen (DO) level, an agitation rate, and a pH in the one or more fermenters;
supplying a carbon source feeding solution to any of the one or more fermenters individually, whenever condition(s) (i) and/or (ii) are met:
(i) said DO level exceeds above about 35% to 45% and said agitation rate exceeds about 300 to 1,000 rpm;
(ii) said agitation rate exceeds about 400 to 700 rpm and said pH exceeds about 7.0 to 7.4; and
supplying a nitrogen source to any of the one or more fermenters in which induction of expression of the recombinant protein has been initiated, individually, at about I₀ (time at initiation of induction) or I₁ or both;
optionally, supplying a nitrogen source to any of the one or more fermenters, individually, at one or more time points from about S₅, S₆, S₇, S₈, S₉ and S₁₀.

11. The method according to claim 9, further comprising the step of detecting one or more deamidated Pro-hIFN A2b at positions from Asn at position 46, Gln at position 159, Asn at position 157, and/or Gln at position 102, wherein the step of detection occurs between each individual steps of claim 9, and/or at the end of the final step of claim 9, and wherein the presence of one or more deamidated Pro-hIFN A2b at any positions indicate one or more method steps of claim 9 was utilized.

12. The method according to claim 10, further comprising the step of detecting one or more deamidated Pro-hIFN A2b at positions from Asn at position 46, Gln at position 159, Asn at position 157, and/or Gln at position 102, wherein the step of detection occurs between each individual steps of claim 10, and/or at the end of the final step of claim 10, and wherein the presence of one or more deamidated Pro-hIFN A2b at any positions indicate one or more method steps of claim 10 was utilized.

13. The method according to claim 9, further comprising the step of detecting one or more deamidated Pro-hIFN A2b at positions from Gln at position 21, Gln at position 41, Gln at position 47, Asn at position 66, Asn at position 94, Gln at position 125, Asn at position 46, Gln at position 159, Asn at position 157, and/or Gln at position 102, wherein the step of detection occurs between each individual steps of claim 9, and/or at the end of the final step of claim 9, and wherein the presence of one or more deamidated Pro-hIFN A2b at any positions indicate one or more method steps of claim 9 was utilized.

14. The method according to claim 10, further comprising the step of detecting one or more deamidated Pro-hIFN A2b at positions from Gln at position 21, Gln at position 41, Gln at position 47, Asn at position 66, Asn at position 94, Gln at position 125, Asn at position 46, Gln at position 159, Asn at position 157, and/or Gln at position 102, wherein the step of detection occurs between each individual steps of claim 10, and/or at the end of the final step of claim 10, and wherein the presence of one or more deamidated Pro-hIFN A2b at any positions indicate one or more method steps of claim 10 was utilized.

15. The method according to claim 9, further comprises the step of detecting one or more deamidated Pro-hIFN A2b at positions from Gln at position 21, Gln at position 41, Gln at position 47, Asn at position 66, Asn at position 94, and/or Gln at position 125, wherein the step of detection occurs between each individual steps of claim 9, and/or at the end of the final step of claim 9, and wherein the presence of one or more deamidated Pro-hIFN A2b at any positions indicate one or more method steps of claim 9 was utilized.

16. The method according to claim 10, further comprises the step of detecting one or more deamidated Pro-hIFN A2b at positions from Gln at position 21, Gln at position 41, Gln at position 47, Asn at position 66, Asn at position 94, and/or Gln at position 125, wherein the step of detection occurs between each individual steps of claim 10, and/or at the end of the final step of claim 10, and wherein the presence of one or more deamidated Pro-hIFN A2b at any positions indicate one or more method steps of claim 10 was utilized.

17. The method according to claim 9, further comprising the step of quantifying one or more said deamidated Pro-hIFN.

18. The method according to claim 10, further comprising the step of quantifying one or more said deamidated Pro-hIFN.

19. The method according to claim 17, wherein
(a) the percentage of deamidated species for Asn at position 46 is at least about 92%, the percentage of deamidated species for Asn at position 157 is at least about 2.5%, and/or the percentage of deamidated species for Gln at position 102 is at least about 0.32%;
(b) the percentage of deamidated species for Asn at position 46 is at least about 61.64%, percentage of deamidated species for Asn at position 157 is at least about 17.87%, and/or the percentage of deamidated species for Gln at position 102 is at least about 2.37%; or
(c) the percentage of deamidated species for Asn at position 46 is at least about 15.71%, the percentage of deamidated species for Gln at position 102 is at least about 0.10%, and/or the percentage of deamidated species for Asn at position 157 is at least about 0.54%, and
wherein the presence of one or more deamidated Pro-hIFN A2b at any positions and in any amount listed in (a) to (c) indicate one or more method steps of claim 9 was utilized.

20. The method according to claim 17, wherein
(a) the percentage of deamidated species for Asn at position 46 is at least about 92%, the percentage of deamidated species for Asn at position 157 is at least about 2.5%, and/or the percentage of deamidated species for Gln at position 102 is at least about 0.32%;
(b) the percentage of deamidated species for Asn at position 46 is at least about 61.64%, percentage of deamidated species for Asn at position 157 is at least about 17.87%, and/or the percentage of deamidated species for Gln at position 102 is at least about 2.37%; or
(c) the percentage of deamidated species for Asn at position 46 is at least about 15.71%, the percentage of deamidated species for Gln at position 102 is at least about 0.10%, and/or the percentage of deamidated species for Asn at position 157 is at least about 0.54%, and
wherein the presence of one or more deamidated Pro-hIFN A2b at any positions and in any amount listed in (a) to (c) indicate one or more method steps of claim 10 was utilized.

21. The method according to claim 17, wherein
(a) the percentage of deamidated species for Gln at position 21 is at least about 0.76%, the percentage of deamidated species for Gln at position 41 is at least about 0.02%, the percentage of deamidated species for Gln at position 47 is at least about 0.15%, the percentage of deamidated species for Asn at position 66 is at least about 0.08%, the percentage of deamidated species for Asn at position 94 is at least about 0.11%, the percentage of deamidated species for Gln at position 125 is at least about 0.10%, the percentage of deamidated species for Asn at position 46 is at least about 92%, the percentage of deamidated species for Asn at position 157 is at least about 2.59%, and/or the percentage of deamidated species for Gln at position 102 is at least about 0.32%; or
(b) the percentage of deamidated species for Asn at position 46 is at least about 61.64%, percentage of deamidated species for Asn at position 157 is at least about 17.87%, the percentage of deamidated species for Gln at position 102 is at least about 2.37%, the percentage of deamidated species for Gln at position 21 is at least about 0.12%, the percentage of deamidated species for Gln at position 41 is at about least 0.29%, the percentage of deamidated species for Gln at position 47 is at least about 0.09%, the percentage of deamidated species for Asn at position 66 is at least about 0.06%, the percentage of deamidated species for Asn at position 94 is at least about 0.11%, and/or
the percentage of deamidated species for Gln at position 125 is at least about 0.98%, and wherein the presence of one or more deamidated Pro-hIFN A2b at any positions and in any amount listed in (a) to (b) indicate one or more method steps of claim 9 was utilized.

22. The method according to claim 17, wherein
(a) the percentage of deamidated species for Gln at position 21 is at least about 0.76%, the percentage of deamidated species for Gln at position 41 is at least about 0.02%, the percentage of deamidated species for Gln at position 47 is at least about 0.15%, the percentage of deamidated species for Asn at position 66 is at least about 0.08%, the percentage of deamidated species for Asn at position 94 is at least about 0.11%, the percentage of deamidated species for Gln at position 125 is at least about 0.10%, the percentage of deamidated species for Asn at position 46 is at least about 92%, the percentage of deamidated species for Asn at position 157 is at least about 2.59%, and/or the percentage of deamidated species for Gln at position 102 is at least about 0.32%; or
(b) the percentage of deamidated species for Asn at position 46 is at least about 61.64%, percentage of deamidated species for Asn at position 157 is at least about 17.87%, the percentage of deamidated species for Gln at position 102 is at least about 2.37%, the percentage of deamidated species for Gln at position 21 is at least about 0.12%, the percentage of deamidated species for Gln at position 41 is at about least 0.29%, the percentage of deamidated species for Gln at position 47 is at least about 0.09%, the percentage of deamidated species for Asn at position 66 is at least about 0.06%, the percentage of deamidated species for Asn at position 94 is at least about 0.11%, and/or
the percentage of deamidated species for Gln at position 125 is at least about 0.98%, and
wherein the presence of one or more deamidated Pro-hIFN A2b at any positions and in any amount listed in (a) to (b) indicate one or more method steps of claim 10 was utilized.

23. The method according to claim 18, wherein
(a) the percentage of deamidated species for Gln at position 21 is at least about 0.76%, the percentage of deamidated species for Gln at position 41 is at about least 0.02%, the percentage of deamidated species for Gln at position 47 is at least about 0.15%, the percentage of deamidated species for Asn at position 66 is at least about 0.08%, the percentage of deamidated species for Asn at position 94 is at least about 0.11%, and/or the percentage of deamidated species for Gln at position 125 is at least about 0.10%;
(b) the percentage of deamidated species for Gln at position 21 is at least about 0.12%, the percentage of deamidated species for Gln at position 41 is at about least 0.29%, the percentage of deamidated species for Gln at position 47 is at least about 0.09%, the percentage of deamidated species for Asn at position 66 is at least about 0.06%, the percentage of deamidated species for Asn at position 94 is at least about 0.11%, and/or the percentage of deamidated species for Gln at position 125 is at least about 0.98%; or
(c) the percentage of deamidated species for Gln at position 21 is at least about 0.10%, the percentage of deamidated species for Gln at position 41 is at about least 0.20%, the percentage of deamidated species for Gln at position 47 is at least about 0.08%, the percentage of deamidated species for Asn at position 66 is at least about 0.07%, the percentage of deamidated species for Asn at position 94 is at least about 0.14%, and/or the percentage of deamidated species for Gln at position 125 is at least about 0.06%, and
wherein the presence of one or more deamidated Pro-hIFN A2b at any positions and in any amount listed in (a) to (c) indicate one or more method steps of claim 9 was utilized.

24. The method according to claim 18, wherein
(a) the percentage of deamidated species for Gln at position 21 is at least about 0.76%, the percentage of deamidated species for Gln at position 41 is at about least 0.02%, the percentage of deamidated species for Gln at position 47 is at least about 0.15%, the percentage of deamidated species for Asn at position 66 is at least about 0.08%, the percentage of deamidated species for Asn at position 94 is at least about 0.11%, and/or the percentage of deamidated species for Gln at position 125 is at least about 0.10%;
(b) the percentage of deamidated species for Gln at position 21 is at least about 0.12%, the percentage of deamidated species for Gln at position 41 is at about least 0.29%, the percentage of deamidated species for Gln at position 47 is at least about 0.09%, the percentage of deamidated species for Asn at position 66 is at least about 0.06%, the percentage of deamidated species for Asn at position 94 is at least about 0.11%, and/or the percentage of deamidated species for Gln at position 125 is at least about 0.98%; or
(c) the percentage of deamidated species for Gln at position 21 is at least about 0.10%, the percentage of deamidated species for Gln at position 41 is at about least 0.20%, the percentage of deamidated species for Gln at position 47 is at least about 0.08%, the percentage of deamidated species for Asn at position 66 is at least about 0.07%, the percentage of deamidated species for Asn at position 94 is at least about 0.14%, and/or the percentage of deamidated species for Gln at position 125 is at least about 0.06%, and
wherein the presence of one or more deamidated Pro-hIFN A2b at any positions and in any amount listed in (a) to (b) indicate one or more method steps of claim 10 was utilized.

25. A method of detecting whether a manufacturing procedure is used to produce Pro-hIFN alpha 2b in a product, batch product, and/or intermediate thereof, said method comprises
analyzing and/or quantifying the present of one or more deamidated Pro-hIFN A2b in said product, batch product, and/or intermediate thereof.

26. The method according to claim 25, wherein said one or more deamidated Pro-hIFN A2b comprises deamidation at positions Asn at position 46, Gln at position 159, Asn at position 157, and/or Gln at position 102.

27. The method according to claim 25, wherein said one or more deamidated Pro-hIFN A2b comprises deamidated Pro-hIFN A2b at positions Gln at position 21, Gln at position 41, Gln at position 47, Asn at position 66, Asn at position 94, Gln at position 125, Asn at position 46, Gln at position 159, Asn at position 157, and/or Gln at position 102.

28. The method according to claim 25, wherein said one or more Pro-hIFN A2b comprises deamidated Pro-hIFN A2b at positions Gln at position 21, Gln at position 41, Gln at position 47, Asn at position 66, Asn at position 94, and/or Gln at position 125.

29. The method according to claim 26, wherein
(a) the percentage of deamidated species for Asn at position 46 is at least about 92%, the percentage of deamidated species for Asn at position 157 is at least about 2.5%, and/or the percentage of deamidated species for Gln at position 102 is at least about 0.32%;
(b) the percentage of deamidated species for Asn at position 46 is at least about 61.64%, percentage of deamidated species for Asn at position 157 is at least about 17.87%, and/or the percentage of deamidated species for Gln at position 102 is at least about 2.37%; or
(c) the percentage of deamidated species for Asn at position 46 is at least about 15.71%, the percentage of deamidated species for Gln at position 102 is at least about 0.10%, and/or the percentage of deamidated species for Asn at position 157 is at least about 0.54%.

30. The method according to claim 27, wherein
(a) the percentage of deamidated species for Gln at position 21 is at least about 0.76%, the percentage of deamidated species for Gln at position 41 is at least about 0.02%, the percentage of deamidated species for Gln at position 47 is at least about 0.15%, the percentage of deamidated species for Asn at position 66 is at least about 0.08%, the percentage of deamidated species for Asn at position 94 is at least about 0.11%, the percentage of deamidated species for Gln at position 125 is at least about 0.10%, the percentage of deamidated species for Asn at position 46 is at least about 92%, the percentage of deamidated species for Asn at position 157 is at least about 2.59%, and/or the percentage of deamidated species for Gln at position 102 is at least about 0.32%; or
(b) the percentage of deamidated species for Asn at position 46 is at least about 61.64%, percentage of deamidated species for Asn at position 157 is at least about 17.87%, the percentage of deamidated species for Gln at position 102 is at least about 2.37%, the percentage of deamidated species for Gln at position 21 is at least about 0.12%, the percentage of deamidated species for Gln at position 41 is at about least 0.29%, the percentage of deamidated species for Gln at position 47 is at least about 0.09%, the percentage of deamidated species for Asn at position 66 is at least about 0.06%, the percentage of deamidated species for Asn at position 94 is at least about 0.11%, and/or
the percentage of deamidated species for Gln at position 125 is at least about 0.98%.

31. The method according to claim 28, wherein
(a) the percentage of deamidated species for Gln at position 21 is at least about 0.76%, the percentage of deamidated species for Gln at position 41 is at about least 0.02%, the percentage of deamidated species for Gln at position 47 is at least about 0.15%, the percentage of deamidated species for Asn at position 66 is at least about 0.08%, the percentage of deamidated species for Asn at position 94 is at least about 0.11%, and/or the percentage of deamidated species for Gln at position 125 is at least about 0.10%;
(b) the percentage of deamidated species for Gln at position 21 is at least about 0.12%, the percentage of deamidated species for Gln at position 41 is at about least 0.29%, the percentage of deamidated species for Gln at position 47 is at least about 0.09%, the percentage of deamidated species for Asn at position 66 is at least about 0.06%, the percentage of deamidated species for Asn at position 94 is at least about 0.11%, and/or the percentage of deamidated species for Gln at position 125 is at least about 0.98%; or
(c) the percentage of deamidated species for Gln at position 21 is at least about 0.10%, the percentage of deamidated species for Gln at position 41 is at about least 0.20%, the percentage of deamidated species for Gln at position 47 is at least about 0.08%, the percentage of deamidated species for Asn at position 66 is at least about 0.07%, the percentage of deamidated species for Asn at position 94 is at least about 0.14%, and/or the percentage of deamidated species for Gln at position 125 is at least about 0.06%.

32. The method according to claim 27, wherein the quantification amount of the deamidated Pro-hIFN alpha 2b at Asn 66:Asn 46 and Gln 125 comprises the deamidated Pro-hIFN alpha 2b at Asn 66:Asn 46 with ratio at about 0.8:1.4:3.8, about 1:1.8:16, and/or about 4:5.5:5.

33. The method according to claim 27, wherein the quantification amount of the deamidated Pro-hIFN alpha 2b at Gln 21, Gln 41, Gln 47, Asn 66:Asn 46 and Gln 125 comprises the deamidated Pro-hIFN alpha 2b at Gln 21, Gln 41, Gln 47, Asn 66:Asn 46, and Gln 125 with ratio at about 1:2:0.8:0.8:1.4:3.8, about 2:4.8:1.5:1.:1.8:16, about 2:4.8:1.5:1.:1.8:16, and/or about 38:1:8:4:5.5:5.

34. The method according to claim 28, wherein the quantification amount of the deamidated Pro-hIFN alpha 2b at Gin 21, Gin 41 and Gin 47 comprises the deamidated Pro-hIFN alpha 2b at Gin 21, Gin 41 and Gin 47 with ratio at about 1:2:0.8, about 2:4.8:1.5, and/or about 38:1:8.
